(19) United States
(12) Patent Application Publication
(10) Pub. No.: US 2007/0196865 A1
(43) Pub. Date: Aug. 23, 2007

(54) Methods Of Screening Compositions For G Protein-Coupled Receptors Aganist Agonists

(75) Inventor: Conrad L. Cowan, Holly Springs, NC (US)

Correspondence Address: DORSEY & WHITNEY LLP, 555 CALIFORNIA STREET, SUITE 1000, SUITE 1000, SAN FRANCISCO, CA, 94104, US

(73) Assignee: Molecular Devices Corporation

(21) Appl. No.: 10/572,529

(22) PCT Filed: Sep. 16, 2004
(86) PCT No.: PCT/US04/30261

§ 371 Date: Mar. 5, 2007

Related U.S. Application Data
(60) Provisional application No. 60/503,447, filed on Sep. 16, 2003.

(51) Int. Cl. G01N 33/567; G01N 33/53

(52) U.S. Cl. 435/7.2

(57) ABSTRACT

Methods of screening compositions for G protein-coupled receptor (“GPCR”) agonist activity against two or more GPCRs in a multiplex receptor assay format are disclosed. One or more cells expressing at least two different GPCRs are exposed to a test composition and it is determined whether or not the composition gives an indication of GPCR agonist activity with respect to any of the at least two different GPCRs. Each of the one or more cells also includes one or more conjugates comprising a marker molecule and a protein associated with the GPCR desensitization pathway of one or more of the GPCRs; that are being used to screen the composition for GPCR agonist activity. The conjugate or conjugates are used to indicate, through the use of the marker molecule, GPCR agonist activity of the test composition with respect to each of the GPCRs that are being used to screen the test composition.

### CROSS-REFERENCE TO RELATED APPLICATIONS

This application claims the benefit of U.S. Provisional Patent Application No. 60/503,447, filed Sep. 16, 2003, the entire content of which is hereby incorporated by reference herein.

### FIELD OF THE INVENTION

The present invention generally relates to methods for screening test compositions for G protein-coupled receptor (“GPCR”) agonist activity, and more particularly relates to screening test compositions for G protein-coupled receptor agonist activity against multiple G protein-coupled receptors (“GPCRs”).

### BACKGROUND OF THE INVENTION

G protein-coupled receptors (GPCRs) are cell surface proteins that translate hormone or ligand binding into intracellular signals. GPCRs are found in all animals, insects, and plants. GPCR signaling plays a pivotal role in regulating various physiological functions including phototransduction, olfaction, neurotransmission, vascular tone, cardiac output, digestion, pain, and fluid and electrolyte balance. Although they are involved in various physiological functions, GPCRs share a number of common structural features. They contain seven membrane domains bridged by alternating intracellular and extracellular loops and an intracellular carboxyl-terminal tail of variable length.

The magnitude of the physiological responses controlled by GPCRs is linked to the balance between GPCR signaling and signal termination. The signaling of GPCRs is controlled by a family of intracellular proteins called arresting. Arrestins bind activated GPCRS, including those that have been agonist-activated and especially those that have been phosphorylated by G protein-coupled receptor kinases (GRKs).

GPCRs have been implicated in a number of disease states, including, but not limited to: cardiac indications such as angina pectoris, essential hypertension, myocardial infarction, supraventricular and ventricular arrhythmias, congestive heart failure, atherosclerosis, renal failure, diabetes, respiratory indications such as asthma, chronic bronchitis, bronchospasm, emphysema, airway obstruction, upper respiratory indications such as rhinitis, seasonal allergies, inflammatory disease, inflammation in response to injury, rheumatoid arthritis, chronic inflammatory bowel disease, glaucoma, hypergastrinemia, gastrointestinal indications such as acid/peptic disorder, erosive esophagitis, gastrointestinal hypersecretion, mastocytosis, gastrointestinal reflux, peptic ulcer, Zollinger-Ellison syndrome, pain, obesity, bulimia nervosa, depression, obsessive-compulsive disorder, organ malformations (for example, cardiac malformations), neurodegenerative diseases such as Parkinson's Disease and Alzheimer's Disease, multiple sclerosis, Epstein-Barr infection and cancer.

Receptors, including GPCRs, have historically been targets for drug discovery and therapeutic agents because they bind ligands, hormones, and drugs with high specificity. Approximately fifty percent of the therapeutic drugs in use today target or interact directly with GPCRs. See, e.g., Jurgen Drews, (2000) “Drug Discovery: A Historical Perspective,” Science 287:1960-1964.

Different assay formats for screening compounds for GPCR activity have been developed. However, some of these existing assays are based on detection of the final cellular response of the specific G_{α} protein subunit (e.g., Gₛ, Gᵢ, and G_{q}) with which a GPCR is associated. Because the final cellular responses of G_{α} protein subunits differ, these existing assays are thus limited to detecting GPCR activity for those receptors associated with specific G_{α} protein subunits. It would be desirable to develop high throughput methods of screening compounds for activity with respect to GPCRs wherein the assay is not dependent on the cellular response of the G_{α} protein subunit with which a GPCR is associated, thus allowing pooling of receptors coupled to different G_{α} protein subunits in a multiplex format.

### SUMMARY OF THE INVENTION

In one embodiment, a method is provided of screening a composition for G protein-coupled receptor (GPCR) agonist activity. A mixture of cells is provided comprising at least a first cell and a second cell. The first cell comprises a first GPCR and a first conjugate of a first marker molecule and an arrestin protein, and the second cell comprises a second GPCR different from the first GPCR and a second conjugate of a second marker molecule and an arrestin protein, with the second conjugate being the same or different from the first conjugate. The mixture of cells is exposed to a test composition and, through detection of the marker molecules in the first and second conjugates, it is determined whether or not the composition gives an indication of GPCR agonist activity with respect to the first or second GPCRs.

In another embodiment, another method of screening a composition for G protein-coupled receptor (GPCR) agonist activity is provided. A cell is provided comprising a first GPCR, a second GPCR different from the first GPCR, a first conjugate of a marker molecule and an arrestin protein associated with the desensitization pathway of the first GPCR, and a second conjugate of a marker molecule and an arrestin protein associated with the desensitization pathway of the second GPCR, with the second conjugate being the same or different from the first conjugate. The cell is exposed to a test composition, and, through detection of the marker molecules in the first and second conjugates, it is determined whether or not the composition gives an indication of GPCR agonist activity with respect to the first or second GPCRS.

In yet another embodiment, yet another method of screening a composition for G protein-coupled receptor (GPCR) agonist activity is provided. A mixture of cells is provided comprising at least a first cell and a second cell. The first cell comprises a first GPCR conjugated to a first marker molecule and the second cell comprises a second GPCR that is different from the first GPCR and is conjugated to a second marker molecule, with the second marker molecule being the same or different from the first marker molecule. The mixture of cells is exposed to a test composition, and it is determined, through detection of the first and second marker molecules, whether or not the composition gives an indication of GPCR agonist activity with respect to the first or second GPCRS.

In a further embodiment, a further method of screening a composition for G protein-coupled receptor (GPCR) agonist activity is provided. A cell is provided comprising a first GPCR conjugated to a first marker molecule and a second GPCR that is different from the first GPCR and is conjugated to a second marker molecule, with the second marker molecule being the same or different from the first marker molecule. The cell is exposed to a test composition, and it is determined, through detection of the first and second marker molecules, whether or not the composition gives an indication of GPCR agonist activity with respect to the first or second GPCRs.

### BRIEF DESCRIPTION OF THE DRAWINGS

FIG. 1 illustrates an example of a desensitization pathway of a GPCR in response to an agonist. Reference numerals in FIG. 1 correspond to items depicted therein as follows: cell membrane-1; GPCR or GPCR-marker molecule conjugate-2; carboxyl terminal tail of GPCR-3; extracellular region-4; intracellular region/cytosol-5; arrestin protein or arrestin-marker molecule conjugate-6; GPCR-arrestin protein complex-7; clathrin-coated pit/vesicle-8; endosome-9; agonist for GPCR-10; third intracellular loop-11; intramembrane portion of GPCR-12; G protein-coupled receptor kinase (GRK)-15; G protein-20.

FIG. 2 is an illustrative, non-limiting list of known GPCRs that may be used with the present invention.

FIG. 3 is an illustrative, non-limiting list of known receptors, including the amino acid sequence for their carboxyl terminal tails (SEQ ID NOS: 1-39) and appropriate classification. For the Class B receptor examples, the residues that may function as phosphorylation sites in the enhanced affinity motifs are shown in bolded italics.

4 is a list of amino acid and nucleic acid sequences of the following GPCRs that have been modified to have enhanced affinity for arrestin: hGPR3-Enhanced receptor, hGPR6-Enhanced receptor, hGPR12-Enhanced receptor, hSREB3-Enhanced receptor, hSREB2-Enhanced receptor, hGPR8-Enhanced receptor, and hGPR22-Enhanced receptor. FIGS. 4A and 4B respectively illustrate the amino acid sequence (SEQ ID NO: 40) and the nucleic acid sequence (SEQ ID NO: 41) of the hGPR3-Enhanced receptor. FIGS. 4C and 4D respectively illustrate the amino acid sequence (SEQ ID NO.: 42) and the nucleic acid sequence (SEQ ID NO: 43) of the hGPR6-Enhanced receptor. FIGS. 4E and 4F respectively illustrate the amino acid sequence (SEQ ID NO: 44) and the nucleic acid sequence (SEQ ID NO: 45) of the hGPR12-Enhanced receptor. FIGS. 4G and 4H respectively illustrate the amino acid sequence (SEQ ID NO: 46) and the nucleic acid sequence (SEQ ID NO: 47) of the hSREB3-Enhanced receptor. FIGS. 41 and 4J respectively illustrate the amino acid sequence (SEQ ID NO: 48) and the nucleic acid sequence (SEQ ID NO: 49) of the hSREB2-Enhanced receptor. FIGS. 4K and 4L respectively illustrate the amino acid sequence (SEQ ID NO: 50) and the nucleic acid sequence (SEQ ID NO: 51) of the hGPR8-Enhanced receptor. FIGS. 4M and 4N respectively illustrate the amino acid sequence (SEQ ID NO: 52) and the nucleic acid sequence (SEQ ID NO: 53) of the hGPR22-Enhanced receptor.

FIG. 5 lists GPCRs that have been modified to have enhanced affinity for arrestin.

FIG. 5A shows the amino acid sequence, termed SEQ ID NO: 54, of the β₂AR-V2R chimera.

FIG. 5B shows the amino acid sequence, termed SEQ ID NO: 55, of the MOR-V2R chimera.

FIG. 5C shows the amino acid sequence, termed SEQ ID NO: 56, of the D1AR-V2R chimera.

FIG. 5D shows the amino acid sequence, termed SEQ ID NO: 57, of the 5HT1AR-V2R chimera.

FIG. 5E shows the amino acid sequence, termed SEQ ID NO: 58, of the β3AR-V2R chimera.

FIG. 5F shows the amino acid sequence, termed SEQ ID NO: 59, of the Edg1R-V2R chimera.

FIGS. 6A-6E illustrate concentration-response curves of the average amount of fluorescent intensity of identified “grains” of arrestin-GFP localization (i.e., Fgrains) in individual cell lines expressing α_{1b}AR (FIG. 6A), β2AR (FIG. 6B), AT_{1A}R (FIG. 6C), DOR (FIG. 6D), and V2R (FIG. 6E) after addition of the indicated concentrations of the compounds norepinephrine (NE), angiotensin II (AT), isoproterenol (Iso), [D-Pen2,D-Pen5]-enkephalin (DPDPE), and arginine vasopressin (AVP) (FIG. 6E only) to the individual cell lines as described in Example 1 below.

FIG. 7 illustrates concentration-response curves of the average amount of fluorescent intensity of identified “grains” of arrestin-GFP localization (i.e., Fgrains) in a multiplex assay using a pool of cells from three cell lines expressing β₂AR, AT_{1A}R, and DOR, respectively, after exposing the pooled cells to Iso, AT, and DPDPE as described in Example 1 below.

FIG. 8 illustrates concentration-response curves of the average amount of fluorescent intensity of identified “grains” of arrestin-GFP localization (i.e., Fgrains) in a multiplex assay using a pool of cells from three cell lines expressing β₂AR, AT_{1A}R, and DOR, respectively, after exposing the pooled cells to Iso, clenbuterol (Clen), and albuterol (Alb) as described in Example 1 below.

FIG. 9 illustrates concentration-response curves of the average amount of fluorescent intensity of identified “grains” of arrestin-GFP localization (i.e., Fgrains) in a multiplex assay using a pool of cells from four cell lines expressing α_{1b}AR, β₂AR, AT_{1A}R, and DOR, respectively, after exposing the pooled cells to Iso, AT, NE, and DPDPE as described in Example 1 below.

FIG. 10 illustrates concentration-response curves of the average amount of fluorescent intensity of identified “grains” of arrestin-GFP localization (i.e., Fgrains) in a multiplex assay using a pool of cells from five cell lines expressing α_{lb}AR, β₂AR, AT_{1A}R, DOR, and V2R, respectively, after exposing the pooled cells to Iso, AT, NE, DPDPE, and AVP as described in Example 1 below.

FIGS. 11 and 12 are tables illustrating the results of individual and multiplex assays testing compounds from the LOPAC 640 library to determine whether the compounds have GPCR agonist activity as described in Example 2 below. The compounds were screened in assays against three cell lines expressing human α_{1b}AR, ββ₂AR and DOR, respectively, both individually and in a multiplex format where all three cell lines were pooled. FIG. 11 lists only those compounds exhibiting agonist activity in the individual and/or multiplexed assays are listed. FIG. 12 lists the subset of adrenergic agonists from the LOPAC 640 library. The responses indicating GPCR agonist activity in FIGS. 11 and 12 are listed in bold type.

FIG. 13 illustrates the results of a “spotting” experiment described in Example 3 below in which varying concentrations of isoproterenol, angiotensin, and norepinephrine were randomly distributed in a blinded fashion across an area of a 384 well plate with a pool of three stable cell lines expressing α_{1b}AR, β₂AR, and AT_{1A}R, respectively in each well. FIG. 13 is a representation of a portion of the 384 well plate, where each number represents a response value assigned to an individual well as a result of the assay. Wells to which one of the test agonists were added are enclosed in a box, and wells with a response to the individual compounds greater than three times the standard deviation (used as an indication of GPCR agonist activity for this experiment) are listed in FIG. 13 as follows: isoproterenol=*, angiotensin=**, and norepinephrine=***.

### DETAILED DESCRIPTION OF THE INVENTION

The present invention relates to methods of screening compositions for GPCR agonist activity. Prior to describing this invention in further detail, however, the following terms will first be defined.

### DEFINITIONS

“Arrestin” means all types of naturally occurring and engineered variants of arrestin, including, but not limited to, visual arrestin (sometimes referred to as Arrestin 1), cone arrestin (sometimes referred to as arrestin-4), β-arrestin 1 (sometimes referred to as Arrestin 2), and β-arrestin 2 (sometimes referred to as Arrestin 3). “Arrestin” also includes biologically active fragments of arrestin.

“Biologically active fragment” of an arrestin means a fragment of arrestin that has the ability to bind a wild-type and/or modified GPCR.

An “antibody” is any immunoglobulin, including antibodies and fragments thereof, that bind a specific epitope.

“Carboxyl-terminal tail” means the carboxyl-terminal tail of a GPCR following membrane span 7. The carboxyl-terminal tail of many GPCRs begins shortly after the conserved NPXXY motif that marks the end of the seventh transmembrane domain (i.e. what follows the NPXXY motif is the carboxyl-terminal tail of the GPCR). The carboxyl-terminal tail may be relatively long (approximately tens to hundreds of amino acids), relatively short (approximately tens of amino acids), or virtually non-existent (less than approximately ten amino acids). As used herein, “carboxyl-terminal tail” shall mean all three variants (whether relatively long, relatively short, or virtually non-existent), and may or may not contain palmitoylated cysteine residue(s).

“Marker molecule” means any molecule capable of detection by spectroscopic, photochemical, biochemical, immunochemical, electrical, radioactive, or optical means, including but not limited to, fluorescence, phosphorescence, bioluminescence, or radioactive decay. Marker molecules include, but are not limited to, GFP, luciferase, β-galactosidase, rhodamine-conjugated antibody, and the like. Marker molecules include radioisotopes, epitope tags, affinity labels, enzymes, fluorescent groups, chemiluminescent groups, and the like. Marker molecules include molecules that are directly or indirectly detected as a function of their interaction with other molecule(s).

“GFP” means Green Fluorescent Protein, which refers to various naturally occurring forms of GFP that may be isolated from natural sources or genetically engineered, as well as artificially modified GFPs. GFPs are well known in the art. See, for example, U.S. Pat. Nos. 5,625,048; 5,777,079; and 6,066,476. It is well understood in the art that GFP is readily interchangeable with other fluorescent proteins, isolated from natural sources or genetically engineered, including but not limited to, yellow fluorescent proteins (YFP), red fluorescent proteins (RFP), cyan fluorescent proteins (CFP), blue fluorescent proteins, luciferin, UV excitable fluorescent proteins, or any wave-length in between.

“Downstream” means toward a carboxyl-terminus of an amino acid sequence, with respect to the amino-terminus.

“Upstream” means toward an amino-terminus of an amino acid sequence, with respect to the carboxyl-terminus.

“GPCR” means G protein-coupled receptor and includes GPCRs naturally occurring in nature, as well as GPCRs that have been modified, including the GPCRs described in U.S. patent application Ser. No. 09/993,844.

“Desensitized GPCR” means a GPCR that presently does not have ability to respond to agonist and activate conventional G protein signaling.

“Sensitized GPCR” means a GPCR that presently has ability to respond to agonist and activate conventional G protein signaling.

“GPCR desensitization pathway” means any cellular component of the GPCR desensitization process, as well as any cellular structure implicated in the GPCR desensitization process and subsequent processes, including but not limited to, arrestin, GRKs, GPCRs, AP-2 protein, clathrin, protein phosphatases, and the like.

“GPCR signaling” means GPCR induced activation of G proteins. This may result in, for example, cAMP production.

“G protein-coupled receptor Idnase” (GRK) includes any kinase that has the ability to phosphorylate a GPCR.

An “overexpressed” protein refers to a protein that is expressed at levels greater than wild-type expression levels.

“Unknown Receptor” or “Orphan Receptor” means a GPCR whose endogenous ligand(s) is/are unknown.

“GPCR agonist activity” of a composition (e.g., compound, solution, etc.) is the ability of the composition to stimulate the GPCR desensitization process or a portion thereof.

“Agonist” includes both full and partial agonists.

An “indication” of GPCR agonist activity means evidence of such activity.

“Test composition” or “composition” means any solution, compound, or other substance (including, but not limited to, small molecules such as deoxyribonucleotide and ribonucleotide molecules as well as peptides, proteins, and nucleic acids) to be screened according to the methods described herein for GPCR agonist activity.

“Desensitization” or “GPCR desensitization” refers generally to the process by which sensitized GPCRs are converted to desensitized GPCRs.

Methods of Screening Compositions Using Multiplex Receptor Assay

The methods of the present invention involve screening a test composition for an indication of GPCR agonist activity against two or more GPCRs in a multiplex receptor assay format. That is, using the methods of the present invention, a test composition may be simultaneously screened for GPCR agonist activity against a pool of two or more GPCRs that are different from each other. The methods use the GPCR desensitization process and pathway to detect for indications of GPCR agonist activity, thus allowing the screening of GPCRs that are coupled with different classes of G_{α} protein subunits (that have different final cellular responses) in a common assay format. In some embodiments, the at least two GPCRs (or more than two GPCRs) are coupled with different classes of G_{α} protein subunits.

The methods may be accomplished using one or more cells that together express at least two different GPCRs that are different from one another. The one or more cells may be from one or more stable cell lines expressing one or more GPCRs and/or from one or more cells transiently expressing one or more GPCRs.

The one or more cells expressing the at least two different GPCRs are exposed to a test composition and it is determined whether or not the composition gives an indication of GPCR agonist activity with respect to any of the at least two different GPCRs. The indication of GPCR agonist activity could be specific for each GPCR (i.e., such that any indication of GPCR agonist activity could be attributed to particular GPCRs) or could be nonspecific (i.e., giving an indication that the test composition has GPCR agonist activity with respect to one of the two or more GPCRs, but without being attributable to any particular GPCR(s)). Some embodiments could also include both specific and nonspecific indications of GPCR agonist activity. Detection methods for determining whether there is an indication that a test composition has GPCR agonist activity are discussed below.

Each cell used in the methods expresses (or overexpresses) at least one GPCR that is used to screen the test composition for GPCR agonist activity such that a detection method may be used to determine whether there is an indication of GPCR agonist activity with respect to that specific GPCR when the cell is exposed to the test composition. In some embodiments, a cell may be used that expresses (or overexpresses) two or more GPCRs that are different from each other such that a detection method may be used for determining whether there is an indication that a test composition has GPCR agonist activity with respect to any of (or each of) the different GPCRs. Embodiments of the methods include, but are not limited to, (1) assays using two or more cells each expressing only one type of GPCR that is used for screening a test composition for GPCR agonist activity (where at least two different GPCRs are expressed by the cells), (2) assays using one or more cells each expressing two or more GPCRs for screening against the test composition, and (3) assays using a mixture of the cells used in the assays described in (1) and (2). The cells used in the methods may also contain other GPCRs that are not used for screening the composition.

In addition to containing one or more GPCRs, each cell used in the methods also includes one or more conjugates comprising a marker molecule and a protein associated with the GPCR desensitization pathway of one or more of the GPCRs that are being used in the cell to screen a test composition for GPCR agonist activity. The conjugate or conjugates are used to indicate, through the use of the marker molecule, GPCR agonist activity of a test composition with respect to each of the GPCRs that are being used to screen the test composition. A conjugate may comprise, for example, an arrestin protein and a marker molecule or a GPCR and a marker molecule. Multiple types of conjugates as well as multiple copies of the same type of conjugate may be used in the cells. In addition, in some embodiments, the conjugate(s) may be stably or transiently expressed by the cells used in the methods.

In embodiments using two or more cells expressing two or more different GPCRs, various formats could be used for screening a composition for GPCR agonist activity. In such embodiments, the methods generally comprise exposing the two or more cells to a test composition and determining whether or not the composition gives an indication of GPCR agonist activity with respect to any of the GPCRs. The indication could be nonspecific (i.e., giving the same response regardless of the GPCR) or the indication could be specific for each GPCR by using different marker molecules (that are distinguishable from each other) for the screening of different GPCRS. Indications specific for different GPCRs may be accomplished by, for example, using different cell lines where each cell line expresses different GPCRs and different conjugates having different and distinguishable marker molecules. As an example, an embodiment could use (1) one or more cells from a first cell line expressing a first GPCR and a first conjugate of an arrestin protein and a first marker molecule and (2) one or more cells from a second cell line expressing a second GPCR different from the first GPCR and a second conjugate of an arrestin protein and a second marker molecule that is different and distinguishable from the first marker molecule.

In embodiments using one cell expressing two or more different GPCRs (or embodiments using a plurality of cells expressing the same two or more different GPCRs), various formats could be used for screening a composition for GPCR agonist activity. In such embodiments, the methods generally comprise exposing the cell (or cells) to a test composition and determining whether or not the composition gives an indication of GPCR agonist activity with respect to any of the GPCRs. The indication of GPCR agonist activity may be nonspecific, or measures may be taken such that indications of GPCR agonist activity with respect to different GPCRs in the cell (or cells) may be distinguished (i.e., so that the indication of GPCR agonist activity is specific). For example, separate conjugates could be used for the screening of a test composition with respect to different GPCRs used in the cell such that each conjugate is included only in the desensitization pathway of one of the GPCRs and such that each conjugate includes a different marker molecule that is distinguishable from the other marker molecules upon detection (e.g., a cell could include a first conjugate comprising a first GPCR and a first marker molecule and a second conjugate comprising a second GPCR and a second marker molecule that is different and distinguishable from the first marker molecule).

As mentioned above, the marker molecule(s) of the conjugate(s) in each cell is/are used to provide an indication of whether a test composition has GPCR agonist activity in that particular cell with respect to the GPCR or GPCRs being used in that cell to screen the test composition. Based upon the GPCR desensitization process and pathway, various formats of detection methods may be used as an indication that a test composition has GPCR agonist activity. The format that is used will depend somewhat on the particular protein associated with the desensitization pathway to which the marker molecule is conjugated, as the methods use the GPCR desensitization process and pathway to detect for indications of GPCR agonist activity.

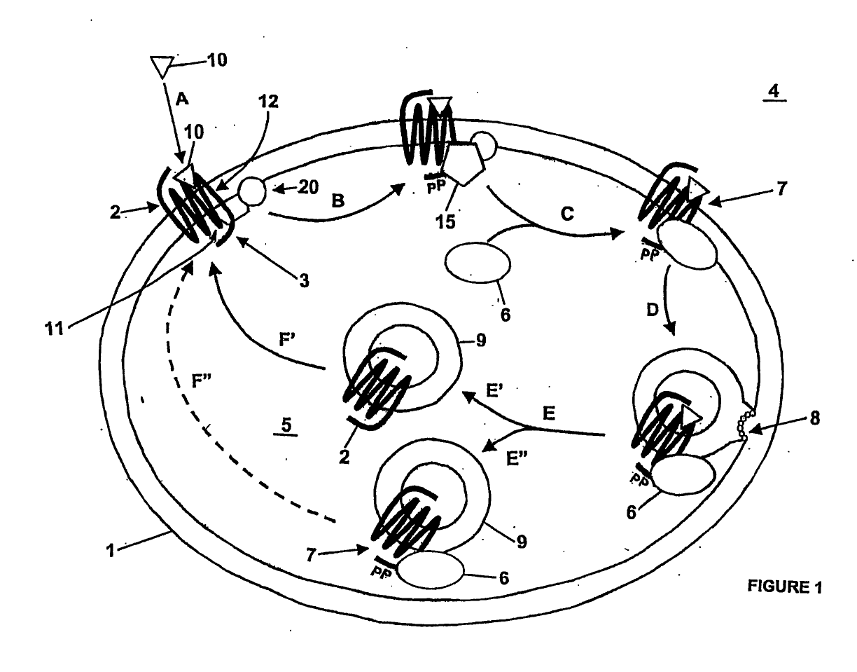

By referring to and describing FIG. 1 (which illustrates an example of a desensitization pathway of a GPCR in response to an agonist), formats of detection methods using conjugates of an arrestin protein and a marker molecule and/or a GPCR and a marker molecule will be better understood. With reference to FIG. 1, after an agonist 10 interacts with a GPCR 2 to activate the GPCR 2 (shown by arrow A), one or more GRKs 15 phosphorylate clusters of serine and threonine residues located in the third intracellular loop 11 or the carboxyl-terminal tail 3 of the GPCR 2 (shown by arrow B). After phosphorylation, an arrestin protein 6 associates with the GRK-phosphorylated GPCR 2 and uncouples the GPCR 2 from its cognate G protein 20 to terminate GPCR signaling and produce a desensitized GPCR. Translocation of the arrestin 6 to the GPCR 2 is shown by arrow C. After the arrestin 6 binds to the GPCR 2, the arrestin/GPCR complex 7 targets to clathrin-coated pits or vesicles 8 (shown by arrow D) for endocytosis. Internalization of the GPCR 2 alone or the arrestin/GPCR complex 7 with an endosome 9 is shown by arrow E. Arrow E′ shows internalization of the GPCR 2 with an endosome 9. Arrow E″ shows internalization of the arrestin/GPCR complex 7 with an endosome 9. After or during internalization, the GPCR 2 is dephosphorylated and is recycled back to the cell membrane 1 as a resensitized GPCR 2. Recycling of the GPCR 2 that was internalized alone is shown by arrow F′. Recycling of the GPCR 2 that was internalized as the arrestin/GPCR complex 7 is shown by arrow F″.

With reference to FIG. 1, when a conjugate of an arrestin protein and a marker molecule is used in a cell, the detection method could detect for any of the following, each of which would be an indication that the test composition has GPCR agonist activity: (1) translocation of the arrestin conjugate 6 from the cytosol 5 to the cell membrane 1 (i.e., arrow C); (2) localization of the arrestin conjugate 6 at the plasma membrane 1; (3) translocation of the arrestin conjugate 6 from the cell membrane 1 to clathrin coated pits/vesicles 8, endosomes 9, or the cytosol 5 (i.e., arrows D and E); or (4) localization of the arrestin conjugate 6 at clathrin coated pits/vesicles 8, endosomes 9, or the cytosol 5. As another example, when a conjugate of a GPCR and a marker molecule is used in a cell, the detection method could look for any of the following, each of which would be an indication that the test composition has GPCR agonist activity: (1) translocation of the GPCR conjugate 2 from the cell membrane 1 to clathrin coated pits/vesicles 8, endosomes 9, or the cytosol 5 (i.e., arrows D and E); or (2) localization of the GPCR conjugate 2 at clathrin coated pits/vesicles 8, endosomes 9, or the cytosol 5. As yet another example, when both a conjugate of an arrestin protein and a marker molecule and a conjugate of a GPCR and a marker molecule are used in a cell, the detection method could look for any of the items/events listed above as well as for localization of the arrestin conjugate with the GPCR conjugate, which would be an indication that the test composition has GPCR agonist activity.

Detection for each of the items/events discussed above could be conducted at one point in time, over a period of time, at two or more points in time for comparison (e.g., before and after exposure to a test composition), etc. An indication of GPCR agonist activity could be determined by detecting for one or more of the items/events discussed above in a cell or cells exposed to the test composition and comparing the results to those obtained by detecting for the same item(s)/event(s) in a control cell not exposed to the test composition, by comparing the results to a predetermined value, or without reference to a predetermined level or a control cell or cells. Therefore, in addition to using certain items/events as indications of GPCR agonist activity, an increase in the level of any of the same items/events discussed above after exposure to a test composition could be used as an indication of GPCR agonist activity of the test composition. Detecting for an increase in the level of the items/events discussed above (e.g., as compared to a control cell or cells not being exposed to the test composition, as compared to a predetermined level, or as compared to a level before exposure to the test composition) may be useful in some embodiments.

When the methods give a nonspecific indication of GPCR agonist activity for a test composition, the result of the assay may be deconvoluted to determine the GPCR(s) for which the composition had GPCR agonist activity. Such deconvolution may be accomplished, for example, by screening the composition individually against each GPCR used in the method that gave an indication of GPCR agonist activity (e.g., by separately exposing cells expressing only one of the GPCRs used in the method to the composition).

The methods may be used to screen a plurality of compositions for GPCR agonist activity against a plurality of GPCRs in a high-throughput manner. For example, a library of compounds could be screened, one compound at a time, against a plurality of GPCRs. As discussed above, the results of the high-throughput screen could give a non-specific indication of GPCR agonist activity and/or could give a specific indication of GPCR agonist activity. If needed, any assay giving an indication that a composition had GPCR agonist activity could be deconvoluted to determine the particular GPCR(s) in the assay for which the composition had GPCR agonist activity.

G Protein-Coupled Receptors (GPCRs)

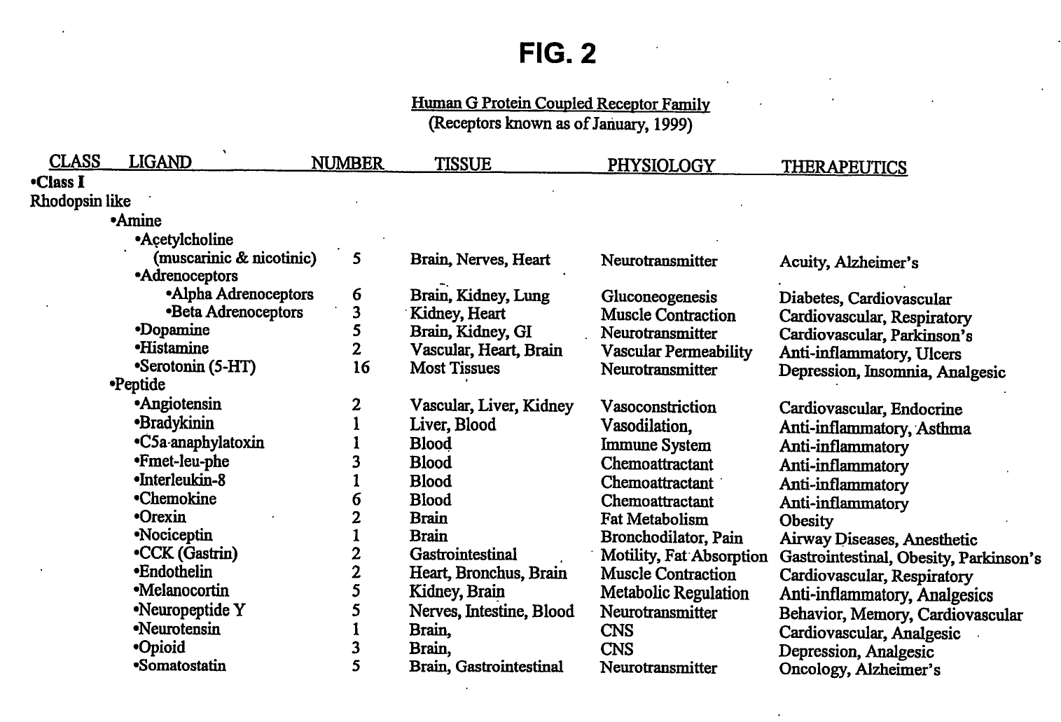

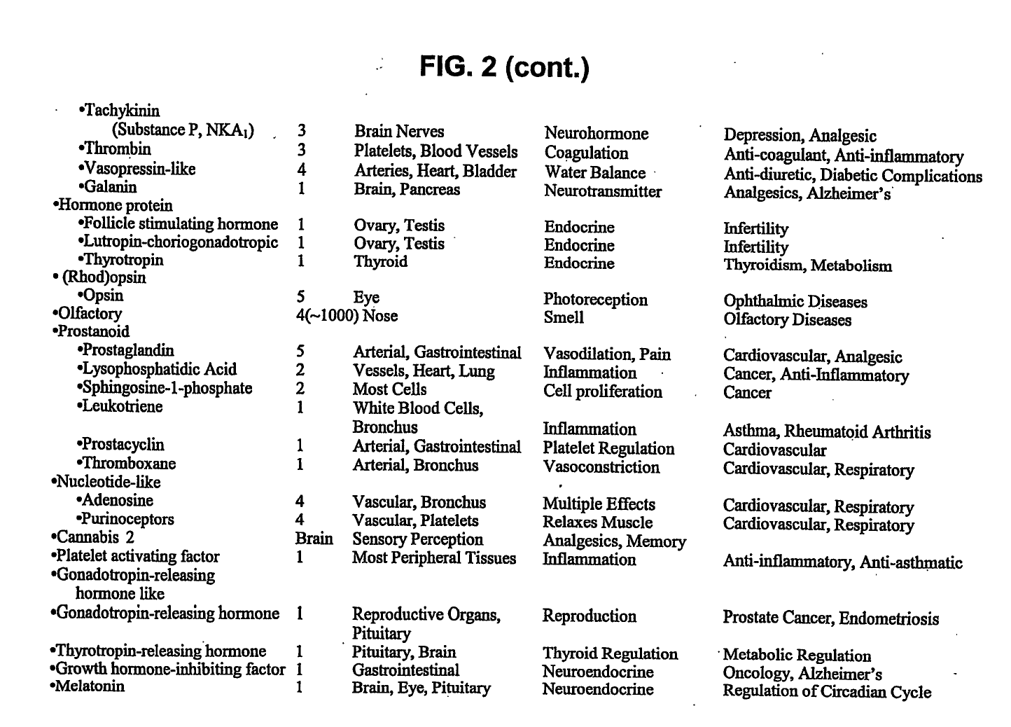

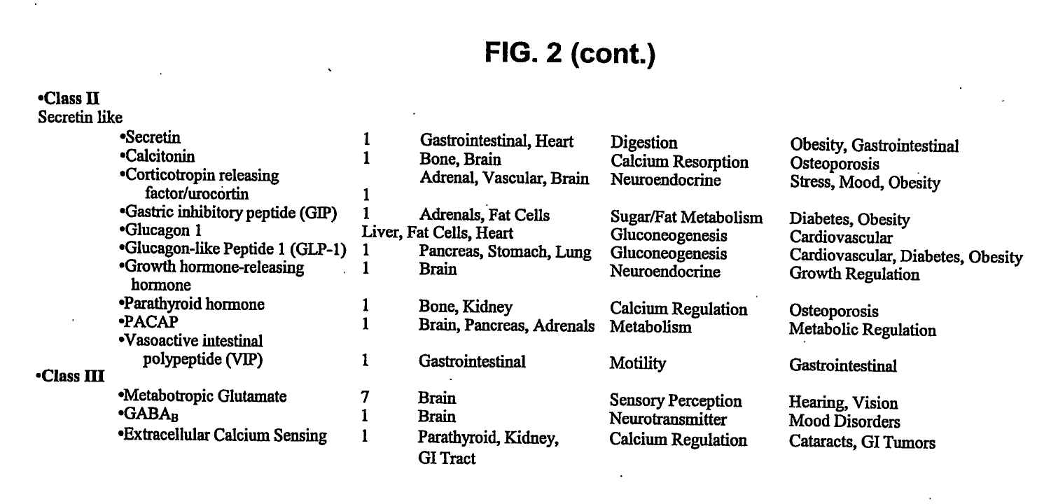

Any G protein-coupled receptor (GPCR) may be used in the methods of the present invention that is capable of participating in the GPCR desensitization process and pathway such that GPCR agonist activity of a test composition may be determined. An illustrative, non-limiting list of known GPCRs with which the present invention may be used is contained in FIG. 2. The receptors are grouped according to classical divisions based on structural similarities and ligands. GPCRs that may be used in the present invention include known GPCRs, unknown or orphan GPCRs, and climeric or modified GPCRs (described more fully below). Modified GPCRs include GPCRs that have one or more modifications in the carboxyl-terminal tail, modifications in the intracellular loop(s), and/or in the cytoplasmic end of the transmembrane region.

By way of example, three major classes of GPCRs for known receptors have been identified: Class A receptors, Class B receptors, and receptors with virtually non-existent carboxyl-terminal tails. The receptors are classified accordingly based on their interactions with an affinity for rat β-arrestin-2 in HEK-293 cells and may be predicted based on the amino acid residues in their carboxyl-terminal tail and the length of their carboxyl-terminal tail. A Class B receptor is a GPCR that has one or more sites of phosphorylation (e.g., clusters of phosphorylation sites) properly positioned in its carboxyl-terminal tail such that it does recruit rat β-arrestin-2 to endosomes in HEK-293 cells under conditions as described in U.S. Pat. No 5,891,646, Oakley, et al. “Differential Affinities of Visual Arrestin, βArrestin1, and βArrestin2 for G Protein-coupled Receptors Delineate Two Major Classes of Receptors,” Journal of Biological Chemistry, Vol 275, No. 22, pp 17201-17210, June 2, 2000, and Oakley et al., “Molecular Determinants Underlying the Formation of Stable Intracellular G Protein-coupled Receptor-β-Arrestin Complexes after Receptor Endocytosis,” Journal of Biological Chemistry, Vol. 276, No. 22, pp 19452-19460, 2001, the contents of which are hereby incorporated by reference in their entirety. A Class A receptor is a GPCR that does not have one or more sites of phosphorylation (e.g., clusters of phosphorylation sites) properly positioned in its carboxyl-terminal tail such that it does not recruit rat β-arrestin-2 to endosomes in HEK-293 cells under conditions as described above for Class B receptors. Receptors with virtually non-existent carboxyl-terminal tails include, for example, olfactory and taste receptors.

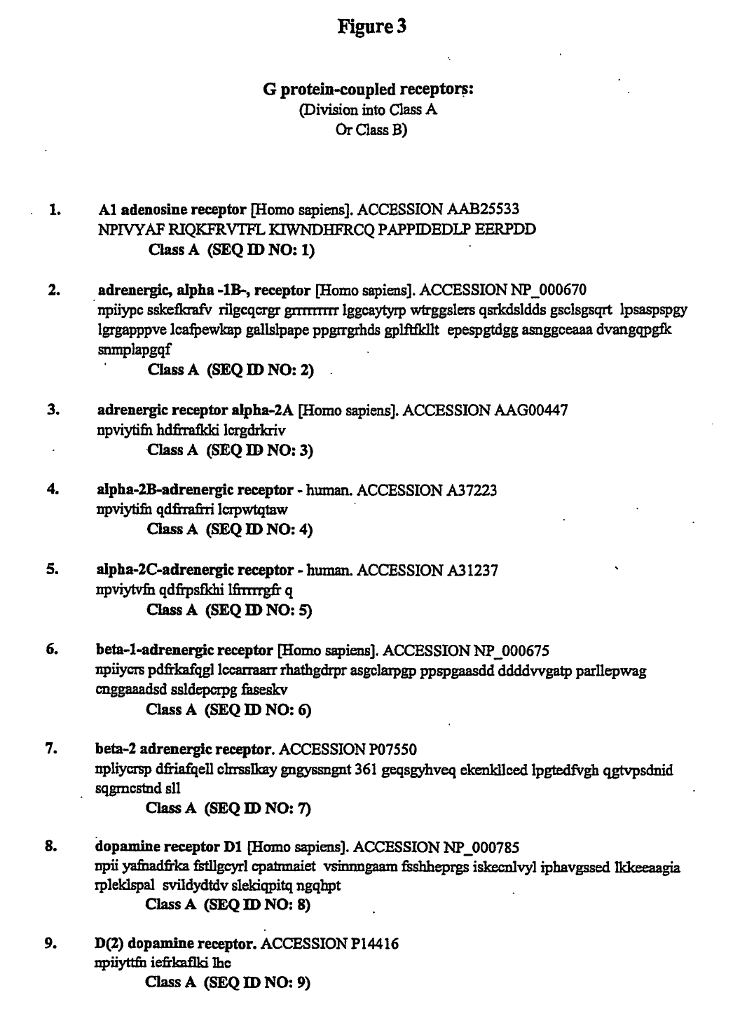

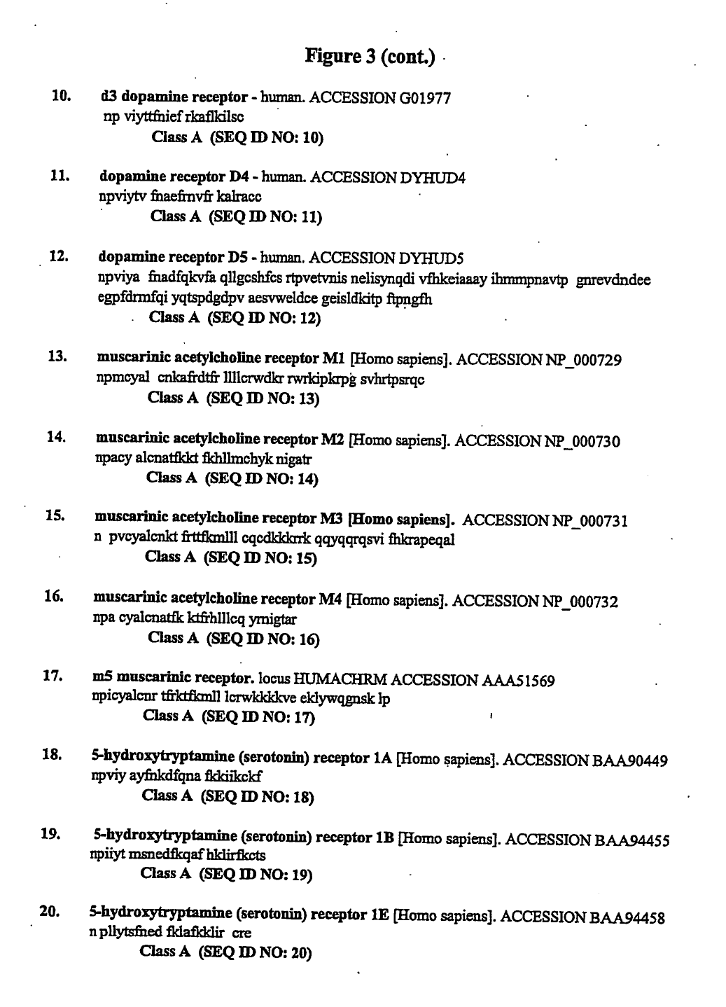

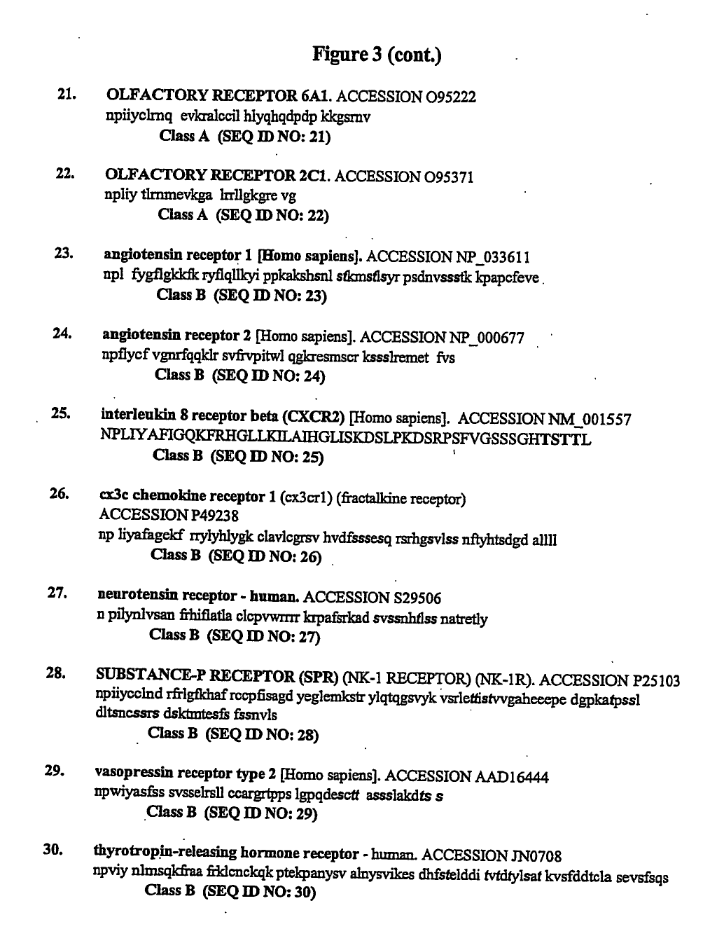

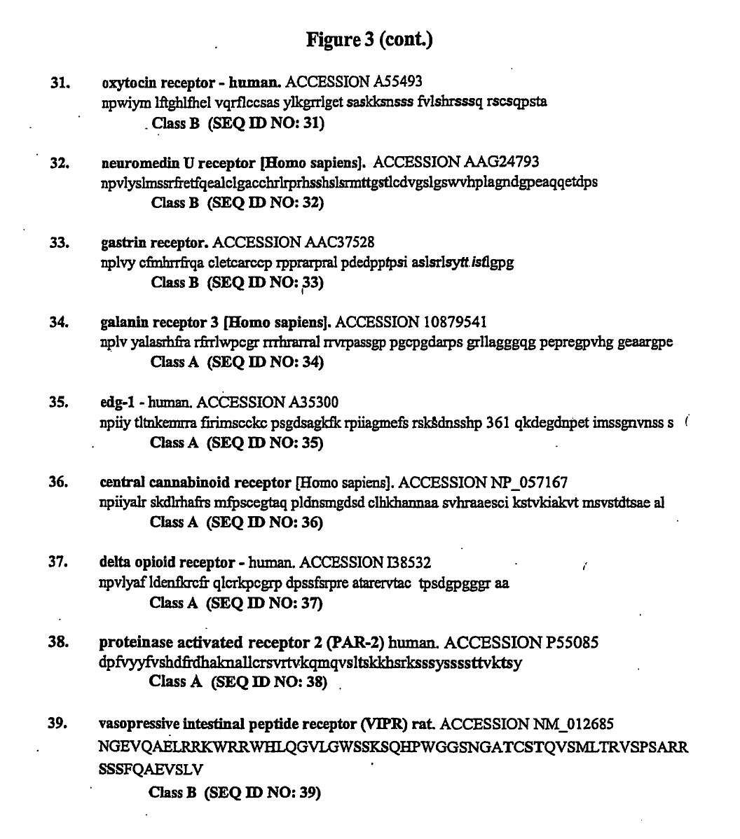

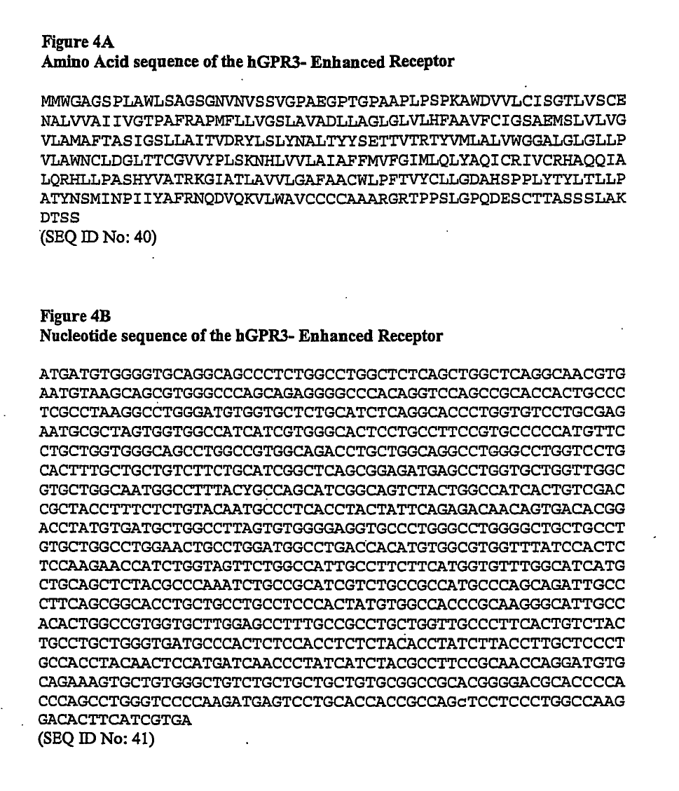

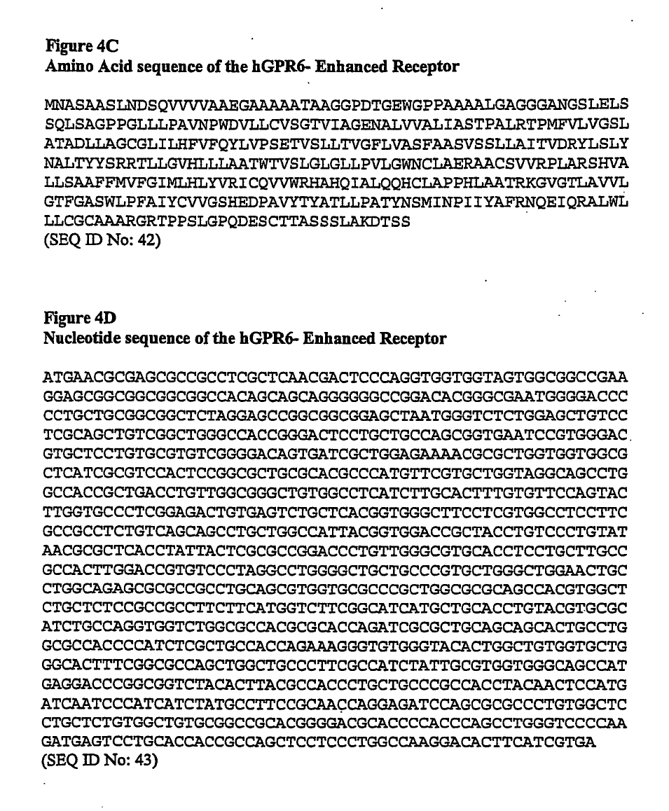

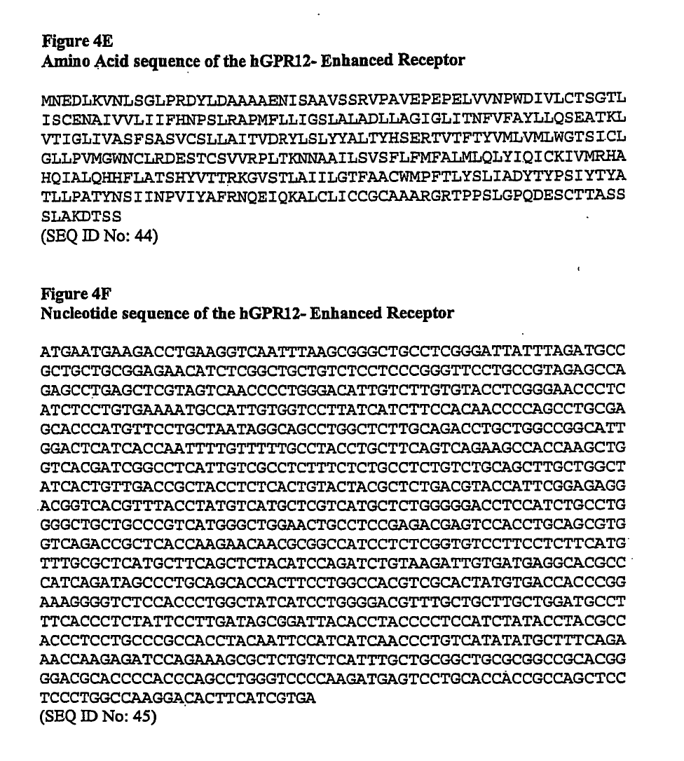

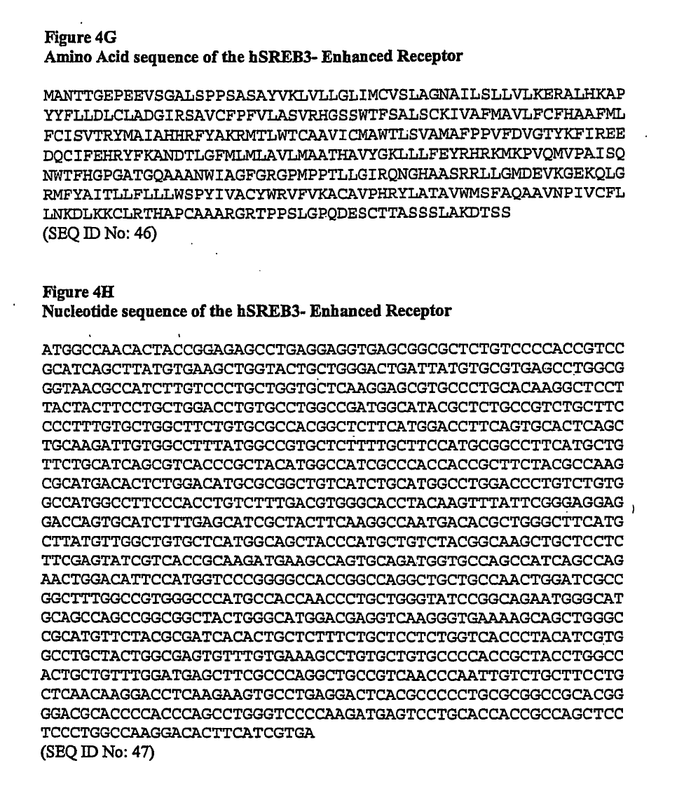

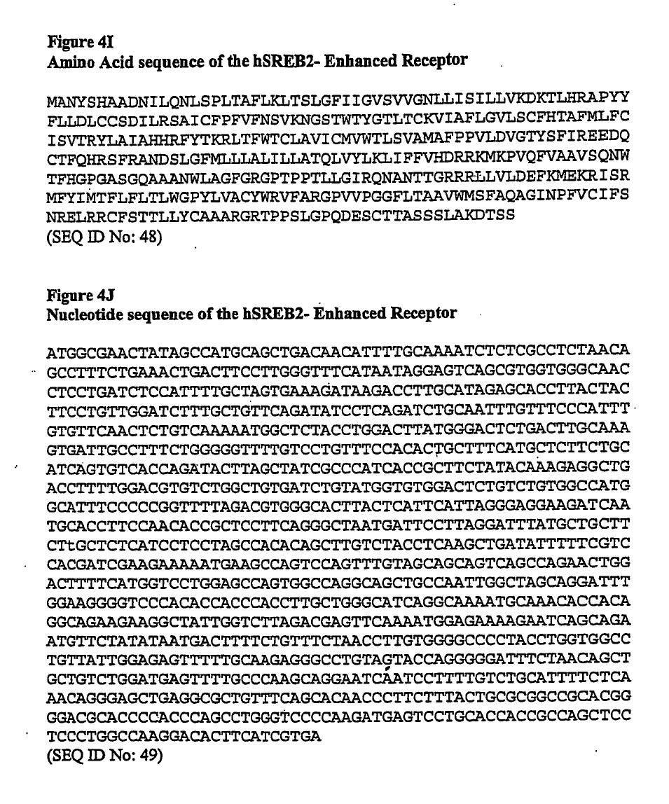

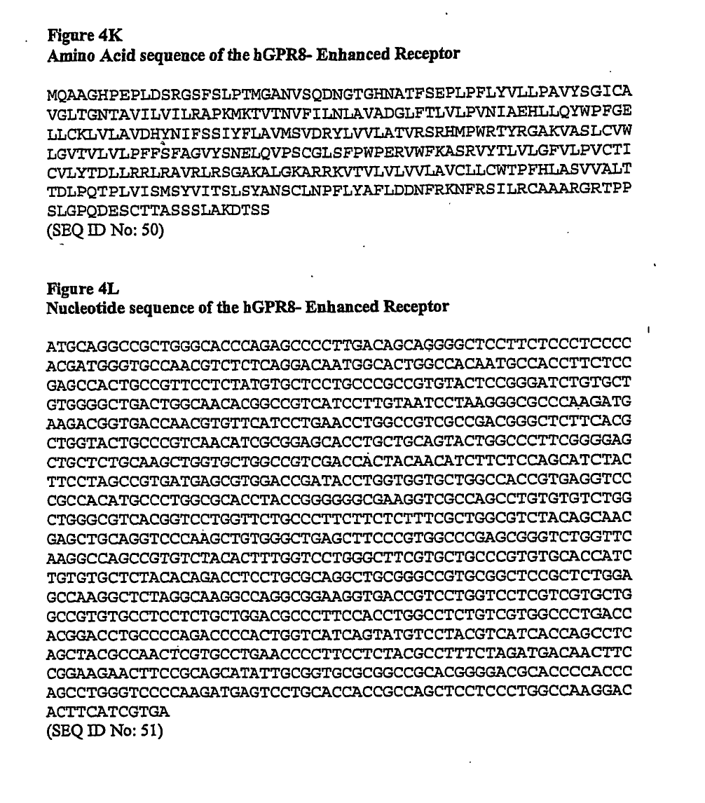

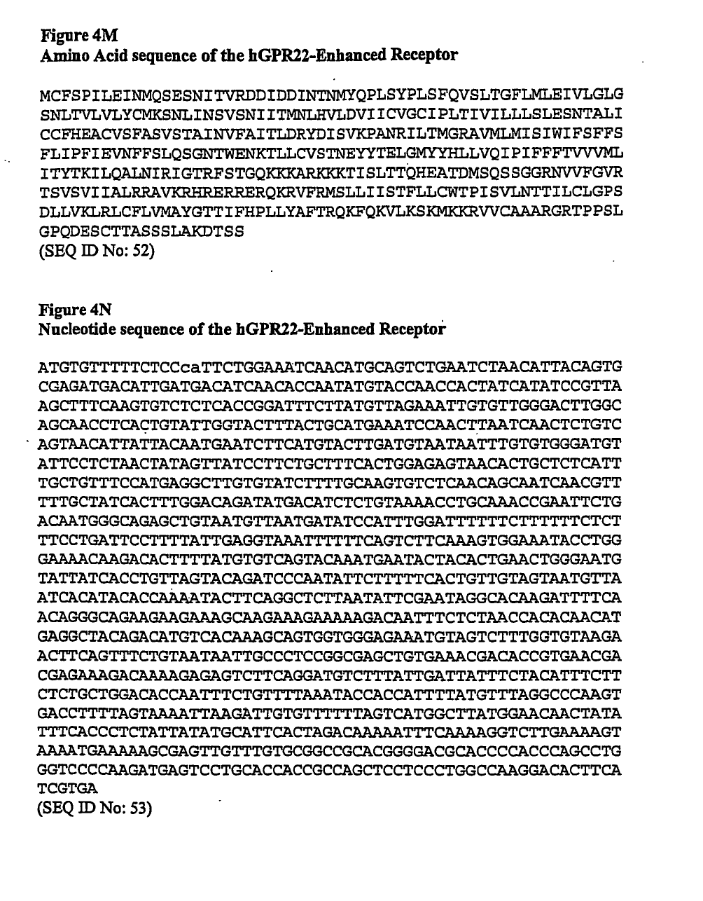

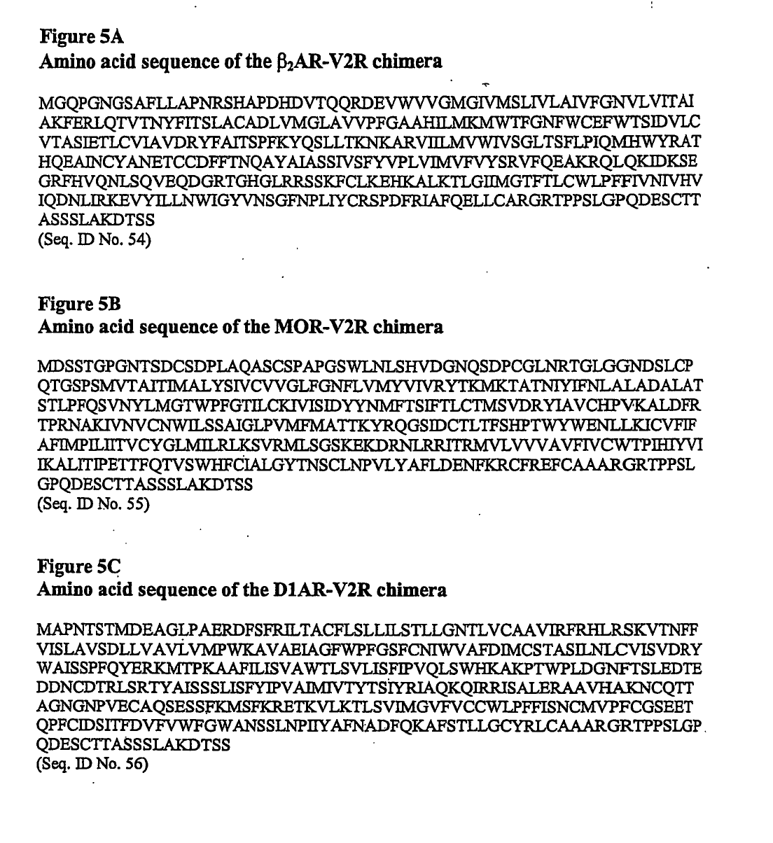

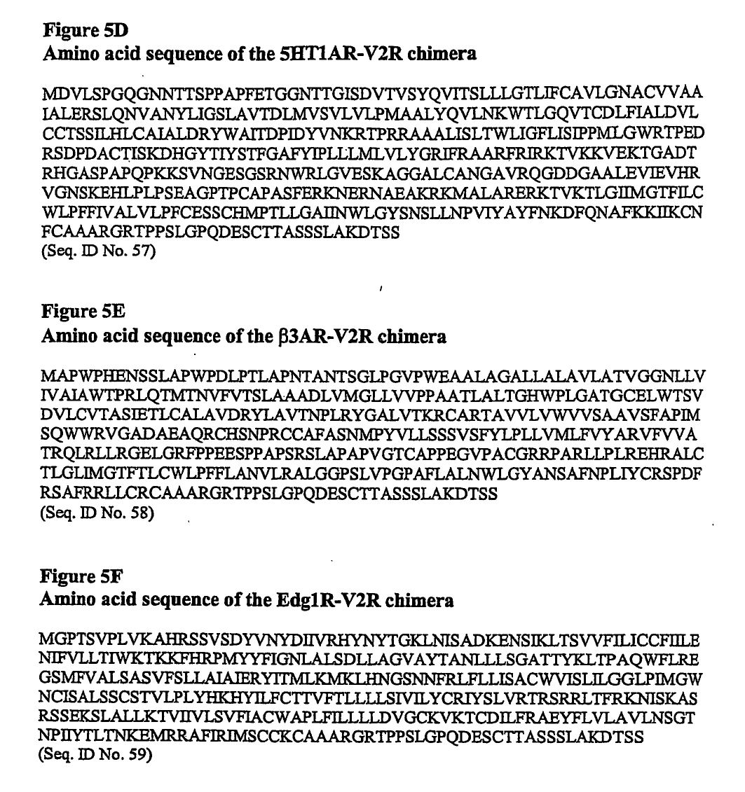

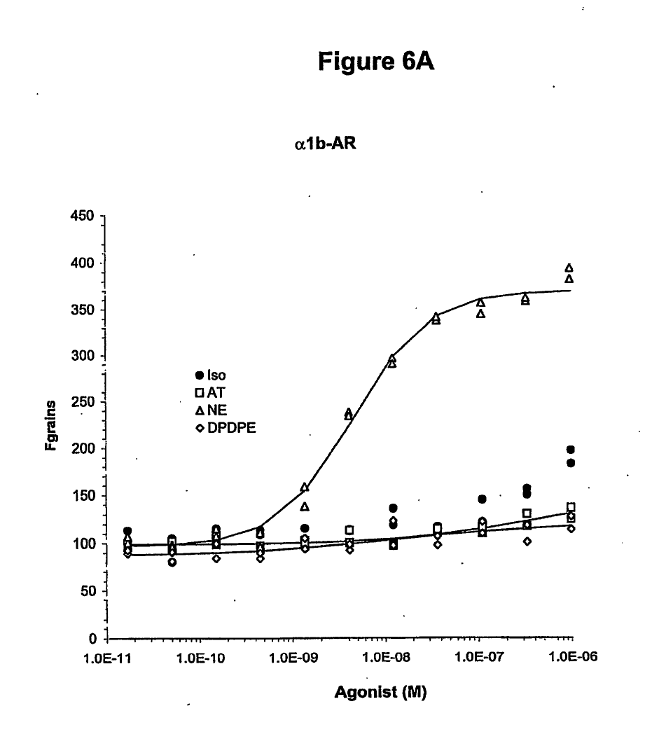

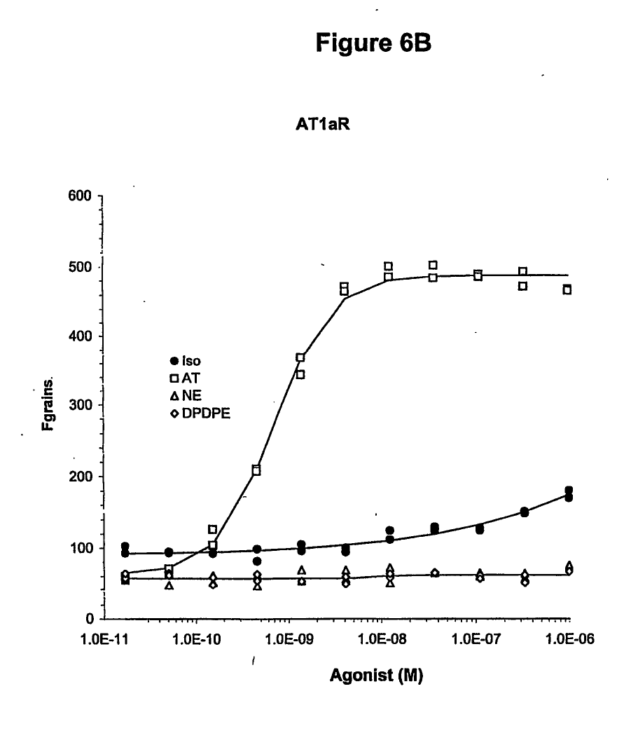

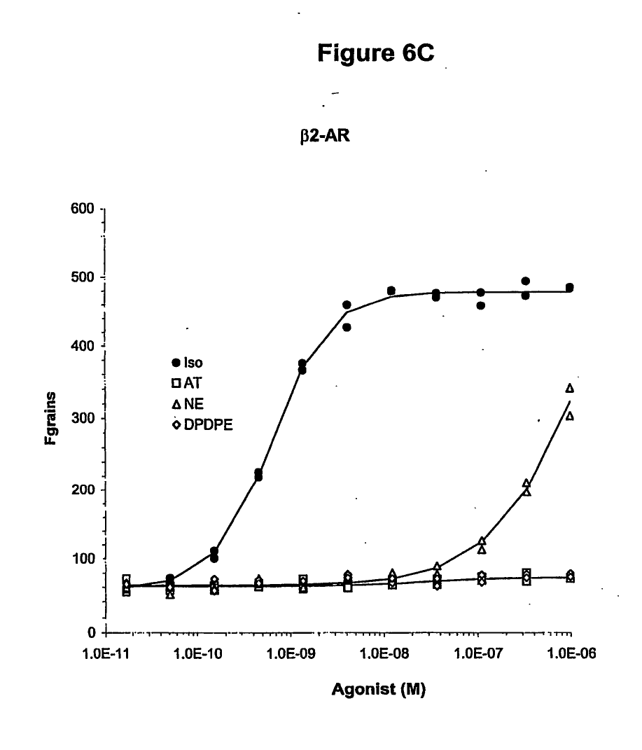

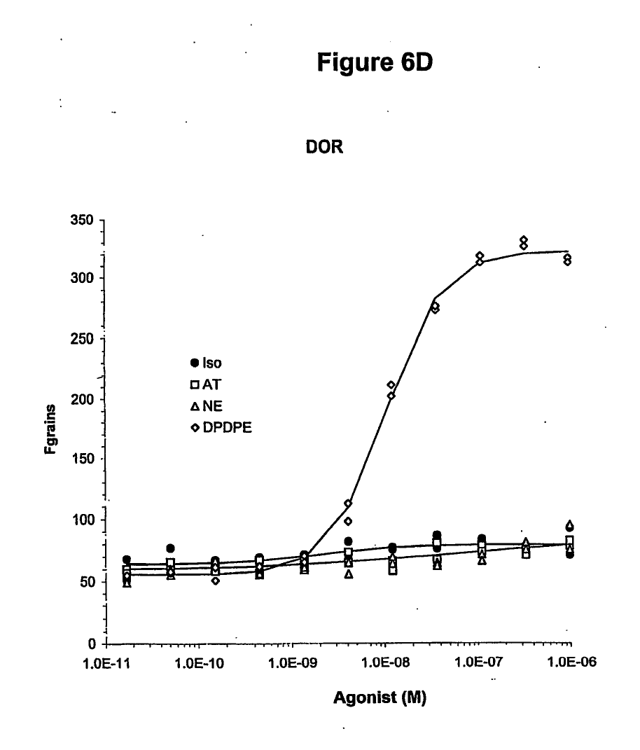

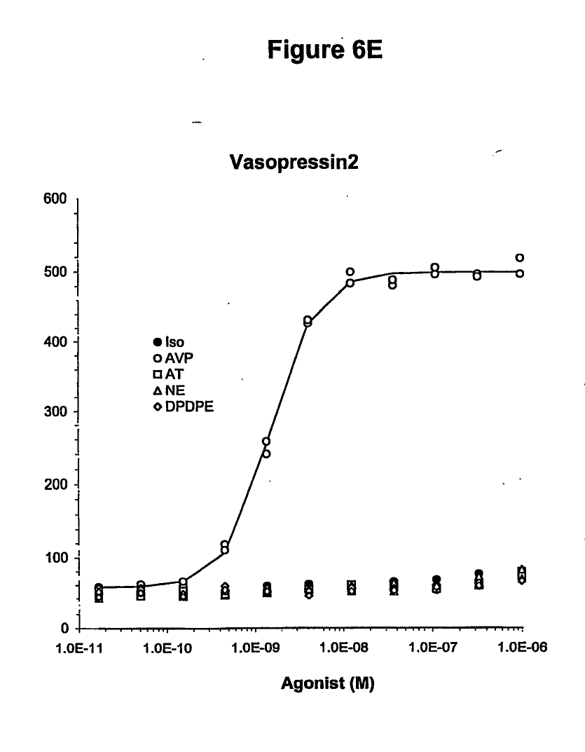

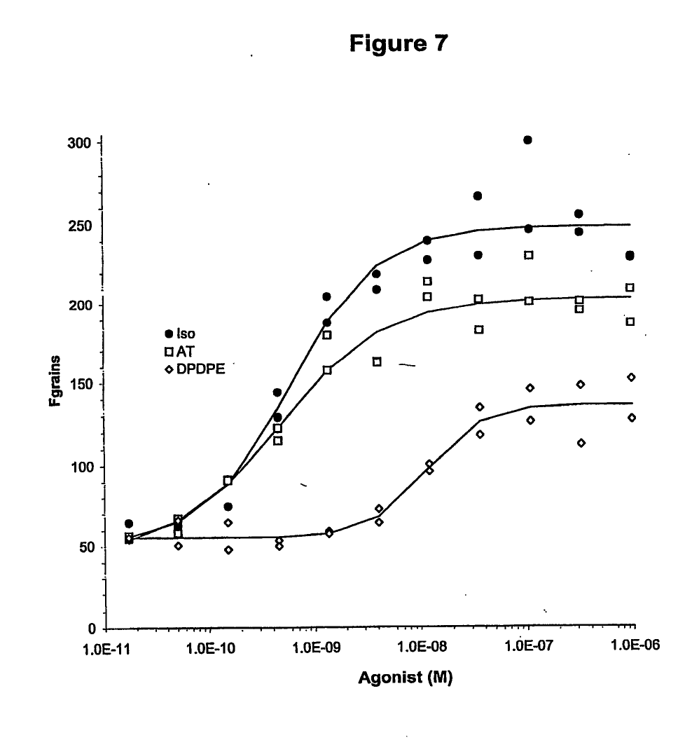

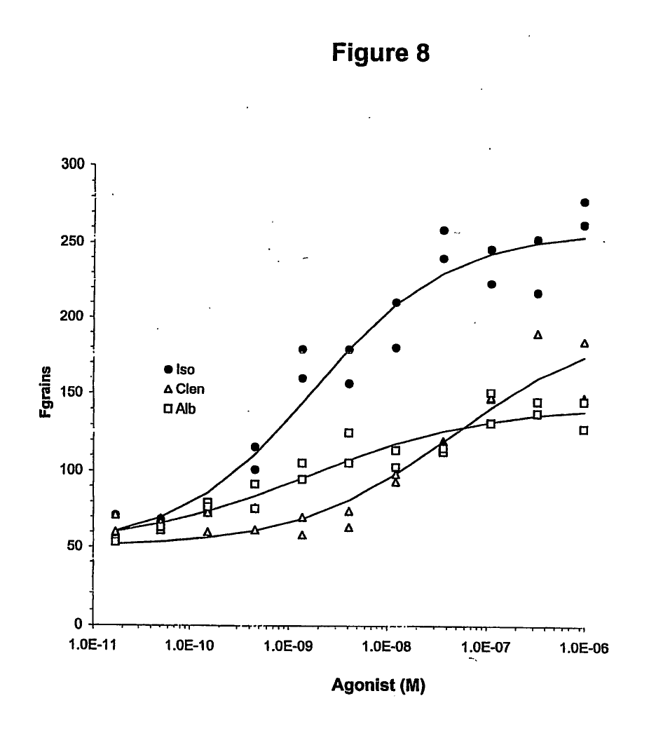

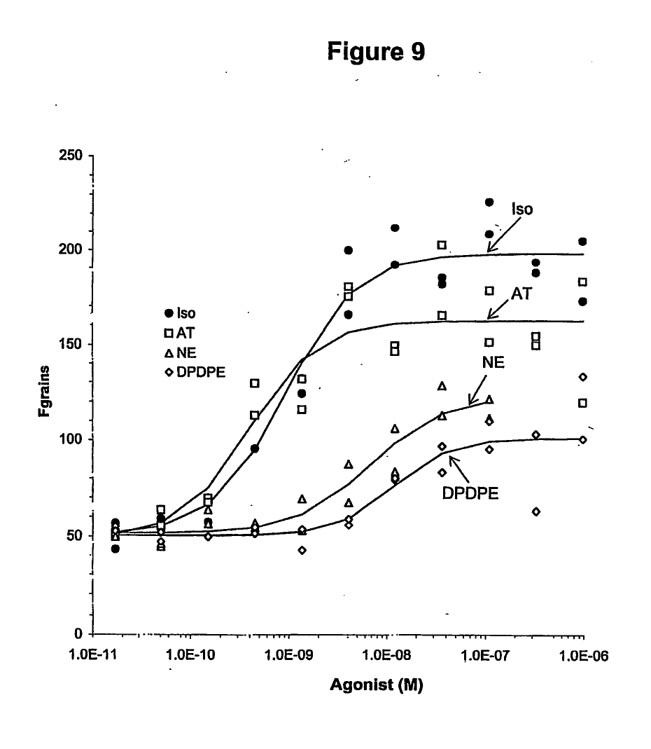

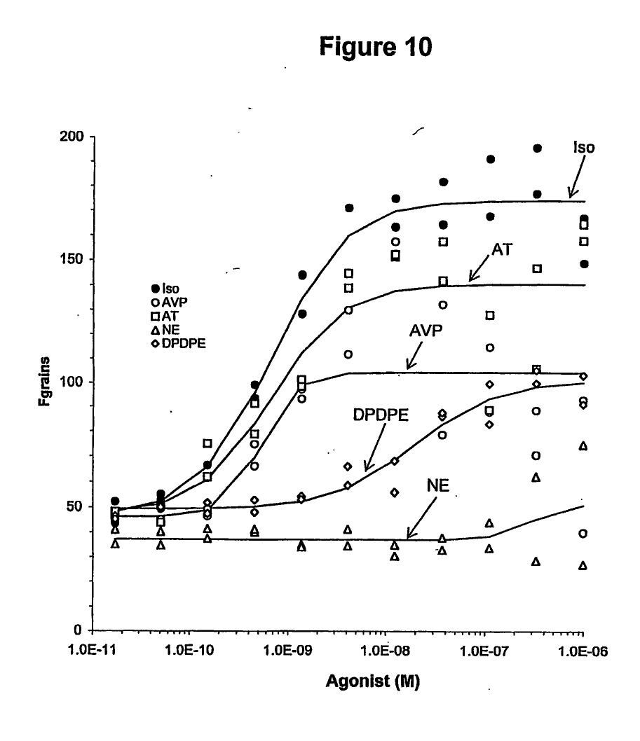

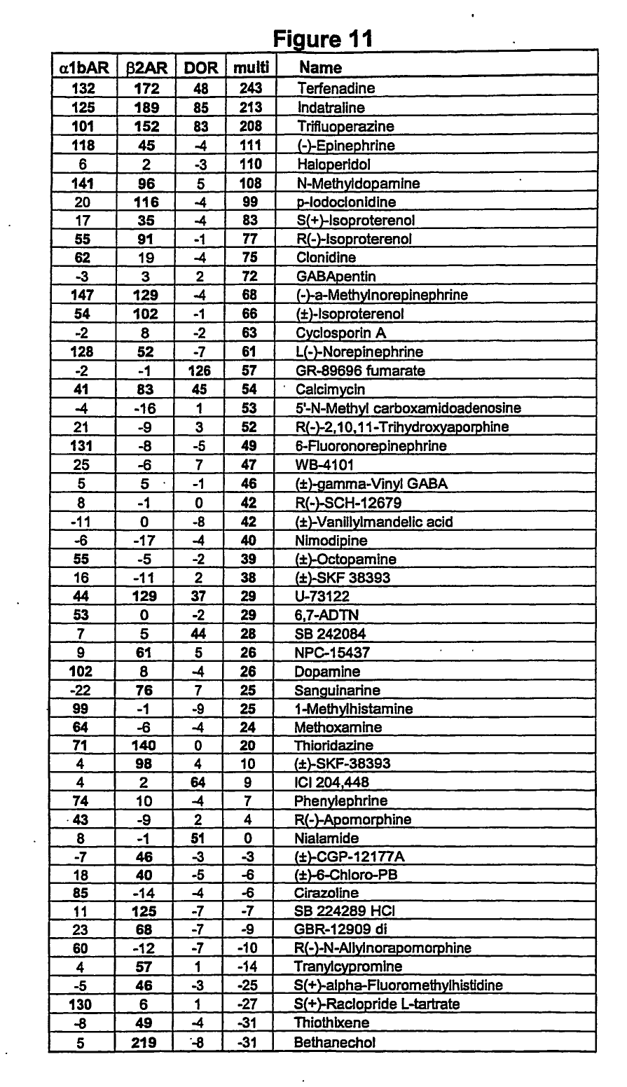

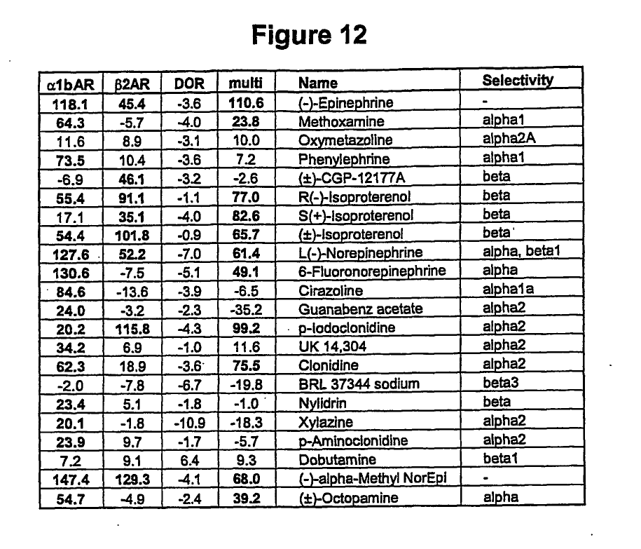

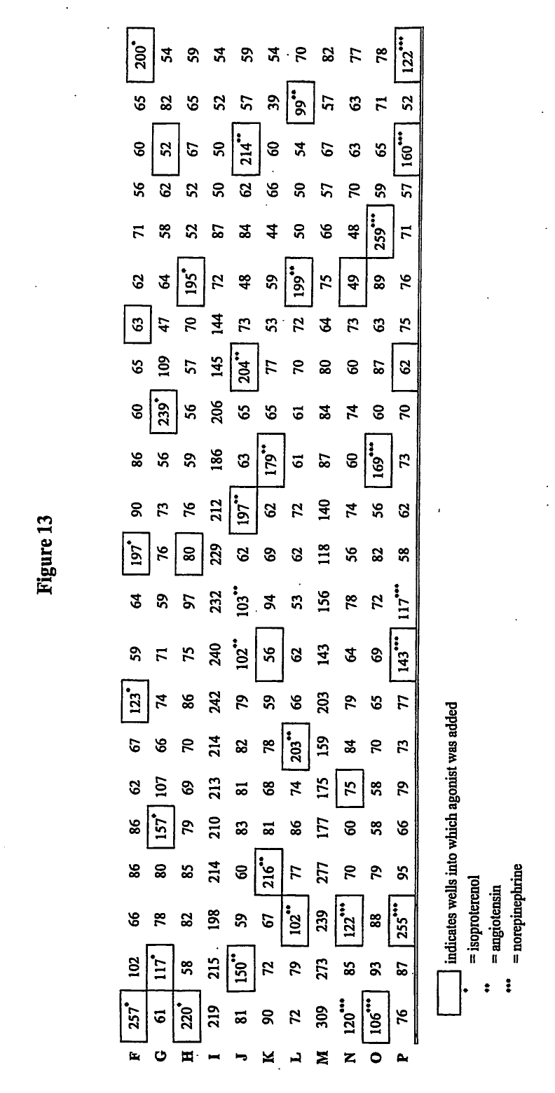

FIG. 3 is an illustrative, non-limiting list of known receptors, including the amino acid sequence for their carboxyl terminal tails and appropriate classification. For the Class B receptor examples, the residues that may function as clusters of phosphorylation sites are shown in bolded italics.

After agonists bind and activate GPCRs, G protein-coupled receptor kinases (GRKs) phosphorylate clusters of serine and threonine residues located in the third intracellular loop or the carboxyl-terminal tail of the GPCRs. After phosphorylation, an arrestin protein associates with the GRK-phosphorylated receptor and uncouples the receptor from its cognate G protein. The interaction of the arrestin with the phosphorylated GPCR terminates GPCR signaling and produces a non-signaling, desensitized receptor.

The arrestin bound to the desensitized GPCR targets the GPCR to clathrin-coated pits for endocytosis by functioning as an adaptor protein, which links the GPCR to components of the endocytic machinery, such as adaptor protein-2 (AP-2) and clathrin. The internalized GPCRs are dephosphorylated and are recycled back to the cell surface resensitized.

The stability of the interaction of arrestin with the GPCR dictates the rate of GPCR dephosphorylation, recycling, and resensitization. When the GPCR has an enhanced affinity for arrestin, the GPCR/arrestin complex is stable, remains intact and is internalized into endosomes. When the GPCR does not have an enhanced affinity for arrestin, the GPCR/arrestin complex tends not to be stable and arrestin is not recruited into endosomes with the GPCR. When the GPCR has an enhanced affinity for arrestin, the GPCR/arrestin complex remains intact, and the GPCR dephosphorylates, recycles and resensitizes slowly. In contrast, GPCRs that dissociate from arrestin at or near the plasma membrane dephosphorylate and recycle rapidly.

The ability of the arrestin to remain associated with the GPCRs is mediated by one or more sites of phosphorylation (e.g., clusters of phosphorylation sites) properly positioned within the carboxyl-terminal tail. These clusters of phosphorylation sites may be serine and threonine residues located in the carboxyl-terminal tail of the GPCR. These clusters are remarkably conserved in their position within the carboxyl-terminal tail domain and serve as primary sites of agonist-dependent phosphorylation.

Modified GPCRs

1. GPCRs With Increased Phosphorylation Sites

GPCRs that do not naturally recruit arrestin to endosomes or do not even naturally recruit arrestin to the plasma membrane may be modified to comprise one or more sites of phosphorylation (e.g., clusters of phosphorylation sites) properly positioned in their carboxyl-terminal tail or properly positioned at other positions in the amino acid sequence (e.g., in the third intracellular loop). This modification allows the modified GPCR to form a stable complex with an arrestin that will internalize into endosomes.

The modified GPCRs that may be used in the methods described herein include GPCRs comprising one or more modifications in their carboxyl-terminal tail. These modifications may comprise inserting one or more sites of phosphorylation (e.g., clusters of phosphorylation sites) within certain regions of the carboxyl-terminal tail, as described in U.S. patent application Ser. No. 09/993,844, filed Nov. 5. 2001, the content of which is hereby incorporated by reference herein in its entirety. As such, the carboxyl-terminal tail may be modified in whole or in part. The carboxyl-terminal tail of many GPCRs begins shortly after a conserved NPXXY motif that marks the end of the seventh transmembrane domain (i.e. what follows the NPXXY motif is the carboxyl-terminal tail of the GPCR). The carboxyl-terminal tail of many GPCRs comprises a putative site of palmitoylation approximately 10 to 25 amino acid residues (e.g., 15 to 20 amino acid residues) downstream of the NPXXY motif. This site is typically one or more cysteine residues. The carboxyl-terminal tail of a GPCR may be relatively long, relatively short, or virtually non-existent. The carboxyl-terminal tail of a GPCR determines the affinity of arrestin binding.

Specific amino acid motifs in the carboxyl-terminal tail promote formation of a stable GPCR/arrestin complex and thus ultimately may promote recruitment of arrestin to endosomes. These amino acid motifs comprise one or more amino acids (e.g., clusters of phosphorylation sites) that may be efficiently phosphorylated and thus readily function as phosphorylation sites. The clusters of amino acids may occupy two out of two, two out of three, three out of three, three out of four positions, four out of four, four out of five positions, five out of five, and the like consecutive amino acid positions. Accordingly, the clusters of amino acids that promote formation of a stable GPCR/arrestin complex are “clusters of phosphorylation sites.” These clusters of phosphorylation sites may be clusters of serine and threonine residues.

GPCRs that form stable complexes with arrestin comprise one or more sites of phosphorylation (e.g., clusters of phosphorylation sites). In addition to the presence of the one or more sites of phosphorylation (e.g., clusters of phosphorylation sites) the sites must be properly positioned within the carboxyl-terminal tail to promote formation of a stable GPCR/arrestin complex. To promote formation of a stable GPCR/arrestin complex, the one or more sites of phosphorylation (e.g., one or more clusters of phosphorylation) may be approximately 15 to 35 (e.g., 15 to 25) amino acid residues downstream of a putative site of palmitoylation of the GPCR. In addition, the one or more sites of phosphorylation (e.g., one or more clusters of phosphorylation, may be approximately 20 to 55 (e.g., 30 to 45) amino acid residues downstream of the NPXXY motif of the GPCR. GPCRs containing one or more sites of phosphorylation (e.g., clusters of phosphorylation sites) properly positioned are typically Class B receptors.

By way of example, the V2R receptor comprises a cluster of phosphorylation sites (SSS) that promotes formation of a stable GPCR/arrestin complex at 19 amino acid residues downstream of the putative site of palmitoylation and 36 amino acid residues downstream of the NPXXY motif The NTR-2 receptor comprises a cluster of phosphorylation sites (STS) that promotes formation of a stable GPCR/arrestin complex at 26 amino acid residues downstream of the putative site of palmitoylation and 45 amino acid residues downstream of the NPXXY motif. The oxytocin receptor (OTR) receptor comprises two clusters of phosphorylation sites (SSLST and STLS) that promote formation of a stable GPCR/arrestin complex, one at 20 amino acid residues downstream of the putative site of palmitoylation and 38 amino acid residues downstream of the NPXXY motif, and the other at 29 amino acid residues downstream of the putative site of palmitoylation and 47 amino acid residues downstream of the NPXXY motif. The substance P receptor (SPR, also known as the neurokinin-1 receptor) comprises a cluster of phosphorylation sites (TTIST) that promotes formation of a stable GPCR/arrestin complex at 32 amino acid residues downstream of the putative site of palmitoylation and 50 amino acid residues downstream of the NPXXY motif.

GPCRs that lack one or more sites of phosphorylation (e.g., clusters of phosphorylation sites) properly positioned within the carboxyl terminal tail form GPCR/arrestin complexes that are less stable and dissociate at or near the plasma membrane. These GPCRs are typically Class A receptors, olfactory receptors, taste receptors, and the like. However, stable GPCR/arrestin complexes may be achieved with GPCRs naturally lacking one or more sites of phosphorylation and having a lower affinity for arrestin by modifying the carboxyl-terminal tails of these receptors. The carboxyl-terminal tails may be modified to include one or more sites of phosphorylation (e.g., one or more clusters of phosphorylation sites) properly positioned within the carboxyl terminal tail.

The modified GPCRs that may be used in the methods described herein include GPCRs that have been modified to have one or more sites of phosphorylation (e.g., one or more clusters of phosphorylation) properly positioned in their carboxyl terminal tails. The polypeptide sequences of the modified GPCRs also include sequences having one or more additions, deletions, substitutions, or mutations. These mutations may be substitution mutations made in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. The GPCRs that may be used in the methods described herein should be considered to include sequences containing conservative changes that do not significantly alter the activity or binding characteristics of the resulting protein.

The modified GPCRs that may be used in the methods described herein include GPCRs containing a NPXXY motif, a putative site of palmitoylation approximately 10 to 25 amino acid residues (e.g., 15 to 20 amino acids) downstream of the NPXXY motif, and a modified carboxyl-terminal tail. The modified carboxyl-terminal tail has one or more sites of phosphorylation (e.g., one or more clusters of phosphorylation sites) such that the phosphorylation sites are approximately 15 to 35 (e.g., 15 to 25) amino acid residues downstream of the putative site of palmitoylation of the modified GPCR. The modified carboxyl-terminal tail may have one or more sites of phosphorylation (e.g., one or more clusters of phosphorylation sites) such that the phosphorylation sites are approximately 20 to 55 (e.g., 30 to 45) amino acid residues downstream of the NPXXY of the modified GPCR.

To create a modified GPCR containing a modified carboxyl-terminus region, a GPCR lacking phosphorylation sites or clusters of phosphorylation sites or with a lower or unknown affinity for arrestin may have one or more additions, substitutions, deletions, or mutations of amino acid residues in its carboxyl-terminal tail. These additions, substitutions, deletions, or mutations are performed such that the carboxyl-terminal tail is modified to comprise one or more sites of phosphorylation (e.g., clusters of phosphorylation sites). By way of example, discrete point mutations of the amino acid residues may be made to provide a modified GPCR. By way of example, three consecutive amino acids may be mutated to serine residues to provide a modified GPCR These mutations are made such that the one or more sites of phosphorylation (e.g., clusters of phosphorylation sites) are properly positioned within the carboxyl terminal tail.

In addition, to create a modified GPCR containing a modified carboxyl-terminal tail region, mutations may be made in a nucleic acid sequence of a GPCR lacking sites of phosphorylation or clusters of phosphorylation sites or with a lower or unknown affinity for arrestin such that a particular codon is changed to a codon that codes for a different amino acid (e.g., a serine or threonine). Such a mutation is generally made by making the fewest nucleotide changes possible. A substitution mutation of this sort can be made to change an amino acid in the resulting protein to create one or more sites of phosphorylation (e.g., clusters of phosphorylation sites). Also by way of example, discrete point mutations of the nucleic acid sequence may be made. The phosphorylation sites are positioned such that they are located approximately 15 to 35 amino acid residues downstream of the putative site of palmitoylation of the modified GPCR.

Furthermore, to provide modified GPCRS, a GPCR lacking properly positioned phosphorylation sites or with a lower or unknown affinity for arrestin may also have its carboxyl-terminal tail, in whole or in part, exchanged with that of a GPCR having properly positioned clusters of phosphorylation sites. The site of exchange may be after or including the conserved NPXXY motif. As an alternative, a putative site of palmitoylation of a GPCR may be identified at approximately 10 to 25 (e.g., 15 to 20) amino acid residues downstream of the conserved NPXXY motif, and the site of exchange may be after or including the palmitoylated cysteine(s). As discussed below, if a putative site of palmitoylation does not exist, one may be introduced in the GPCR. The carboxyl-terminal tail of a GPCR lacking properly positioned clusters of phosphorylation sites or with a lower or unknown affinity for arrestin may be exchanged at an amino acid residue in close proximity to a putative site of palmitoylation. In one embodiment, the carboxyl-terminal tail of a GPCR lacking properly positioned clusters of phosphorylation sites or with a lower or unknown affinity for arrestin is exchanged at a putative site of palmitoylation approximately 10 to 25 (e.g., 15 to 20) amino acid residues downstream of the NPXXY motif, such that the palmitoylated cysteine residue is maintained. The carboxyl-terminal tail of a GPCR lacking properly positioned clusters of phosphorylation sites may be exchanged in a manner allowing the clusters of phosphorylation sites to be properly positioned within the carboxyl-terminal tail of the modified GPCR. The tails may be exchanged and the modified GPCRs may be constructed accordingly by manipulation of the nucleic acid sequence or the corresponding amino acid sequence.

In a further alternative, the carboxyl-tail of a GPCR, for example a GPCR not containing the NPXXY motif, may be predicted from a hydrophobicity plot and the site of exchange may be selected accordingly. Based on a hydrophobicity plot, one of skill in the art may predict a site where it is expected that the GPCR may anchor in the membrane and then predict where to introduce a putative site of palmitoylation accordingly. Using this technique GPCRs having neither a NPXXY motif nor a putative site of palmitoylation may be modified to create a point of reference (e.g. a putative site of palmitoylation). The introduced putative site of palmitoylation may then be used to position a tail exchange.

The carboxyl-terminal tail used for the exchange may be from a second GPCR having one or more properly positioned clusters of phosphorylation sites and having a putative site of palmitoylation approximately 10 to 25 (e.g., 15 to 20) amino acid residues downstream of a NPXXY motif. The tail as identified may be exchanged, after or including the conserved NPXXY motif. As an alternative, a putative site of palmitoylation of a GPCR may be identified at approximately 10 to 25 (e.g., 15 to 20) amino acid residues downstream of the conserved NPXXY motif, and the tail may be exchanged after or including the palmitoylated cysteine(s). The carboxyl-terminal tail of a GPCR having clusters of phosphorylation sites may be exchanged at an amino acid residue in close proximity to a putative site of palmitoylation. In one embodiment, the carboxyl-terminal tail of a GPCR having clusters of phosphorylation sites is exchanged at a putative site of palmitoylation approximately 10 to 25 (e.g., 15 to 20) amino acid residues downstream of the NPXXY motif, such that the portion of the carboxyl-terminal tail containing the clusters of phosphorylation sites begins at the amino acid residue immediately downstream of the palmitoylated cysteine residue. The carboxyl-terminal tail having clusters of phosphorylation sites used for the exchange may have a marker molecule conjugated to the carboxyl-terminus. The tails may be exchanged and the modified GPCRs may be constructed accordingly by manipulation of the nucleic acid sequence or the corresponding amino acid sequence.

In addition, the carboxyl-terminal tail portion used for the exchange may originate from a polypeptide synthesized to have an amino acid sequence corresponding to an amino acid sequence from a GPCR having one or more sites of phosphorylation (e.g., one or more clusters of phosphorylation sites). The synthesized polypeptide may have a putative site of palmitoylation approximately 10 to 25 (e.g., 15 to 20) amino acid residues downstream of a NPXXY motif. The synthesized polypeptide may have one or more additions, substitutions, mutations, or deletions of amino acid residues that does not affect or alter the overall structure and function of the polypeptide.

Furthermore, the carboxyl-terminal tail portion used for the exchange may originate from a naturally occurring polypeptide recognized to have an amino acid sequence corresponding to an amino acid sequence from a GPCR having one or more clusters of phosphorylation sites. The polypeptide may have a putative site of palmitoylation approximately 10 to 25 (e.g., 15 to 20) amino acid residues downstream of a NPXXY motif. The polypeptide may have one or more additions, substitutions, mutations, or deletions of amino acid residues that does not affect or alter the overall structure and function of the polypeptide.

A modified GPCR containing a modified carboxyl-terminus region may be created by fusing a first carboxyl-terminal tail portion of a GPCR lacking properly positioned clusters of phosphorylation sites or with a lower or unknown affinity for arrestin with a second carboxyl-terminal tail portion of a GPCR or polypeptide having one or more clusters of phosphorylation sites. The second GPCR or polypeptide used for the exchange may have a putative site of palmitoylation approximately 10 to 25 (e.g., 15 to 20) amino acid residues downstream of a NPXXY motif. Accordingly, the modified carboxyl-terminus region of the modified GPCR comprises a portion of a carboxyl-terminal tail from a GPCR lacking properly positioned clusters of phosphorylation sites or with a lower or unknown affinity for arrestin fused to a portion of a carboxyl-terminal tail of a GPCR or polypeptide having clusters of phosphorylation sites. The tail of a GPCR lacking properly positioned clusters of phosphorylation sites may be exchanged after or including the conserved NPXXY motif, and fused to a carboxyl-terminal tail containing clusters of phosphorylation sites, after or including the conserved NPXXY motif As an alternative, the tail of a GPCR lacking properly positioned clusters of phosphorylation sites may be exchanged after or including the palmitoylated cysteine(s), and fused to a tail containing clusters of phosphorylation sites, after or including the palmitoylated cysteine(s). The tails may be exchanged and the modified GPCRs may be constructed accordingly by manipulation of the nucleic acid sequence or the corresponding amino acid sequence.

In a further alternative, the carboxyl-tail of a GPCR, for example a GPCR not containing the NPXXY motif, may be predicted from a hydrophobicity plot and exchanged accordingly. The site of exchange may be selected according to the hydrophobicity plot. Based on a hydrophobicity plot, one of skill in the art may predict a site where it is expected that the GPCR may anchor in the membrane and then predict where to introduce a putative site of palmitoylation accordingly. Using this technique GPCRs having neither a NPXXY motif nor a putative site of palmitoylation may be modified to create a point of reference (e.g. a putative site of palmitoylation). The introduced putative site of palmitoylation may be then used to position a tail exchange. After introduction of a putative site of palmitoylation, the resulting tail may be fused with a second carboxyl-terminal tail portion of a GPCR or polypeptide having one or more clusters of phosphorylation sites and having a putative site of palmitoylation approximately 10 to 25 (e.g., 15 to 20) amino acid residues downstream of a NPXXY motif.

The modified carboxyl-terminus region of the modified GPCR may be fused at amino acid residues in close proximity to a putative site of palmitoylation. In one embodiment, the modified carboxyl-terminus region of the modified GPCR is fused such that the portion from the first GPCR with a lower affinity for arrestin comprises amino acid residues from the NPXXY motif through a putative site of palmitoylation approximately 10 to 25 (e.g., 15 to 20) amino acid residues downstream of the NPXXY motif and the portion from the second GPCR having clusters of phosphorylation sites and a putative site of palmitoylation approximately 10 to 25 (e.g., 15 to 20) amino acid residues downstream of a NPXXY motif comprises amino acid residues beginning with an amino acid residue immediately downstream of the putative site of palmitoylation of the second GPCR extending to the end of the carboxyl-terminus. Such a fusion allows the clusters of phosphorylation sites to be properly positioned within the carboxyl-terminal tail and allows the modified GPCR to maintain its structure and ability to function.

By way of example, a Class A receptor or an orphan receptor may have a portion of its carboxyl-terminal tail exchanged with a portion of a carboxyl-terminal tail from a known Class B receptor. Further, receptors having virtually non-existent carboxyl-terminal tails, for example, olfactory receptors and taste receptors, may have a portion of their carboxyl-terminal tails exchanged with a portion of a carboxyl-terminal tail from a known Class B receptor. The Class B receptor tail used for these exchanges may have a marker molecule fused to the carboxyl-terminus.

Modified GPCRs may be generated by molecular biological techniques standard in the genetic engineering art, including but not limited to, polymerase chain reaction (PCR), restriction enzymes, expression vectors, plasmids, and the like. By way of example, vectors, such as a pEArrB (enhanced arrestin binding, described in U.S. patent application Ser. No. 09/993,844), may be designed to enhance the affinity of a GPCR lacking clusters of phosphorylation sites for arrestin. To form a vector, such as a pEArrB vector, PCR amplified DNA fragments of a GPCR carboxyl-terminus, which forms stable complexes with arrestin, may be digested by appropriate restriction enzymes and cloned into a plasmid. The DNA of a GPCR, which is to be modified, may also be PCR amplified, digested by restriction enzymes at an appropriate location, and subcloned into the vector, such as pEArrB. When expressed, the modified GPCR will contain a polypeptide fused to the carboxyl-terminus. The polypeptide will comprise clusters of phosphorylation sites. In one embodiment, the polypeptide originates from the GPCR carboxyl-terminus of a receptor that forms stable complexes with arrestin.

Such modified GPCRs may also occur naturally as the result of aberrant gene splicing or single nucleotide polymorphisms. Such naturally occurring modified GPCRs would be predicted to have modified endocytic targeting.

As shown in FIG. 5A, a portion of a β₂AR, a Class A receptor, may be fused to a portion of a V2R receptor (a Class B receptor). As shown in the figure, the first 341 amino acids of the β₂AR, Met-1 through Cys-341 (a putative site of palmitoylation) may be fused to the last 29 amino acids of the V2R carboxyl-terminus (Ala-343 through Ser-371; Ala-343 is immediately following a palmitoylated cysteine). This fusion properly positions the V2R cluster of phosphorylation sites (SSS) within the modified GPCR's tail.

As shown in FIG. 5B, a portion of a mu opioid receptor (MOR), a Class A receptor, may be fused to a portion of a V2R receptor (a Class B receptor). As shown in the figure, the first 351 amino acids of the MOR, Met-1 through Cys-351 (a palmitoylated cysteine residue), may be fused to the last 29 amino acid of the V2R carboxyl-terminus (Ala-343 through Ser-371; Ala-343 is immediately following a palmitoylated cysteine). This fusion properly positions the V2R cluster of phosphorylation sites (SSS) within the modified GPCR's tail.

Also as shown in FIG. 5C, a portion of a dopamine D1A receptor (D1AR), a Class A receptor, may be fused to a portion of a V2R receptor. As shown in the figure, the first 351 amino acids of the D1AR, Met-1 through Cys-351 (a palmitoylated cysteine) may be fused to the last 29 amino acid of the V2R carboxyl-terminus (Ala-343 through Ser-371; Ala-343 is immediately following a palmitoylated cysteine). This fusion properly positions the V2R cluster of phosphorylation sites (SSS) within the modified GPCR's tail.

Further as shown in FIG. 5D, a portion of a 5-hydroxytryptamine 1A receptor (5HT1AR), a Class A receptor, may be fused to a portion of a V2R receptor (a Class B receptor). As shown in the figure, the first 420 amino acids of the 5HT1AR, Met-1 through Cys-420 (a palmitoylated cysteine) may be fused to the last 29 amino acid of the V2R carboxyl-terminus (Ala-343 through Ser-371; Ala-343 is immediately following a palmitoylated cysteine). This fusion properly positions the V2R cluster of phosphorylation sites (SSS) within the modified GPCR's tail.

As shown in FIG. 5E, a portion of a β3-adrenergic receptor (β3AR), a Class A receptor, may be fused to a portion of a V2R receptor (a Class B receptor). As shown in the figure, the first 363 amino acids of the β3AR, Met-1 through Cys-363 (a palmitoylated cysteine) may be fused to the last 29 amino acid of the V2R carboxyl-terminus (Ala-343 through Ser-371; Ala-343 is immediately following a palmitoylated cysteine). This fusion properly positions the V2R cluster of phosphorylation sites (SSS) within the modified GPCR's tail.

Finally as shown in FIG. 5F, a portion of a endothelial differentiation, sphingolipid GPCR 1 (Edg1R), a Class A receptor, may be fused to a portion of a V2R receptor (a Class B receptor). As shown in the figure, the first 331 amino acids of the Edg1R, Met-1 through Cys-331 (a palmitoylated cysteine) may be fused to the last 29 amino acid of the V2R carboxyl-terminus (Ala-343 through Ser-371; Ala-343 is immediately following a palmitoylated cysteine). This fusion properly positions the V2R cluster of phosphorylation sites (SSS) within the modified GPCR's tail.

The modified GPCRs described in U.S. Provisional Patent Application No. 60/401,698, filed Aug. 7, 2002, the content of which is hereby incorporated by reference herein in its entirety, may also be used in the present invention. The GPCRs described in U.S. Provisional Patent Application No. 60/401,698 include the following receptors that have enhanced affinity for arrestin: hGPR3E, hGPR6E, hGPR12E, hGPR8E, hGPR22E, hSREB2E, and hSREB3E. The “E” stands for “enhanced arrestin binding”. Each of these modified GPCRs contains a properly positioned cluster of phosphorylation sites (SSS) within the modified GPCR's tail. FIGS. 4A-N list the amino acid and nucleic acid sequences for these GPCRs.

As may be shown by standard receptor binding assays, the modified receptors are essentially indistinguishable from their wild-type counterparts except for an increased affinity for arrestin and thus an increased stability of their complex with arrestin and in their ability to traffic with arrestin and in their ability to recycle and resensitize. For example, the modified receptors are appropriately expressed at the membrane and possess similar affinity for agonists or ligands.

2. Other Modified GPCRs

Other modified GPCRs may also be used in the present invention so long as the modified GPCRs are capable of participating in the GPCR desensitization process and pathway such that GPCR agonist activity of a test composition may be determined.

Cells

Cells useful in the present invention include eukaryotic and prokaryotic cells, including, but not limited to, bacterial cells, yeast cells, fungal cells, insect cells, nematode cells, plant cells, and animal cells. Suitable animal cells include, but are not limited to, HEK cells, HeLa cells, COS cells, U20S cells, CHO-KI cells, and various primary mammalian cells.

Cells useful in the present invention include those that express a known GPCR, a variety of known GPCRs, an unknown GPCR, a variety of unknown GPCRs, a modified GPCR, a variety of modified GPCRs, and combinations thereof. A cell that expresses a GPCR is one that contains the GPCR as a functional receptor in its cell membrane. The cells may naturally express the GPCRs, may be genetically engineered to express the GPCRs at varying levels of expression, or may be genetically engineered to inducibly express the GPCRs. As one skilled in the art readily would understand, the cells may be genetically engineered to express GPCRs by molecular biological techniques standard in the genetic engineering art.

In addition, cells useful in the present invention may stably or transiently express the GPCRs, arrestin proteins, and/or conjugates used in the methods described herein. Methods of expressing genes using non-mammalian viruses (e.g., baculoviruses) described in U.S. Pat. Nos. 4,745,051; 4,879,236; 5,348,886; 5,731,182; 5,871,986; 6,281,009; and 6,238,914; may be used in the present methods. The entire contents of U.S. Pat. Nos. 4,745,051; 4,879,236; 5,348,886; 5,731,182; 5,871,986; 6,281,009; and 6,238,914 are hereby incorporated by reference herein in their entirety.

Conjugates

In the methods of the present invention, each of the cells comprises one or more conjugates of a marker molecule and a protein associated with the GPCR desensitization pathway of one or more GPCRs that are being used in the cell to screen a test composition for GPCR agonist activity. For example, one or more of the cells may comprise a conjugate of an arrestin protein and a marker molecule and/or a conjugate of a GPCR and a marker molecule.

All forms of arrestin, both naturally occurring and engineered variants, including but not limited to, visual arrestin, β-arrestin 1 and β-arrestin 2, may be used in the present invention.

Marker molecules that may be used to conjugate with the arrestin include, but are not limited to, molecules that are detectable by spectroscopic, photochemical, radioactivity, biochemical, immunochemical, colorimetric, electrical, or optical means, including, but not limited to, bioluminescence, phosphorescence, and fluorescence. These marker molecules should be biologically compatible molecules and should not compromise the ability of the arrestin to interact with the GPCR system, and the interaction of the arrestin with the GPCR system must not compromise the ability of the marker molecule to be detected. Marker molecules include radioisotopes, epitope tags, affinity labels, enzymes, fluorescent groups, chemiluminescent groups, and the like. Marker molecules include molecules that are directly or indirectly detected as a function of their interaction with other molecule(s) as well as molecules detected as a function of their location or translocation. In some embodiments, the marker molecules are optically detectable marker molecules, including optically detectable proteins, such that they may be excited chemically, mechanically, electrically, or radioactively to emit fluorescence, phosphorescence, or bioluminescence. Optically detectable marker molecules include, for example, beta-galactosidase, firefly luciferase, bacterial luciferase, fluorescein, Texas Red, horseradish peroxidase, alkaline phosphatase, and rhodamine-conjugated antibody. In other embodiments, the optically detectable marker molecules are inherently fluorescent molecules, such as fluorescent proteins, including, for example, Green Fluorescent Protein (GFP).

The marker molecule may be conjugated to the arrestin protein by methods as described in U.S. Pat. Nos. 5,891,646 and 6,110,693. The marker molecule may be conjugated to the arrestin at the front-end, at the back-end, or in the middle. In some embodiments, the marker molecules are molecules that are capable of being synthesized in the cell. The cell can be transfected with DNA so that the conjugate of arrestin and a marker molecule is produced within the cell. As one skilled in the art readily would understand, cells may be genetically engineered to express the conjugate of arrestin and a marker molecule by molecular biological techniques standard in the genetic engineering art.

The GPCRs used in the present invention may also be conjugated with a marker molecule. In some embodiments, the carboxyl-terminus of the GPCR may be conjugated with a marker molecule. A carboxyl-terminal tail conjugated or attached to a marker molecule can be used in a carboxyl-terminal tail exchange to provide a modified GPCR.

If the GPCR is conjugated with a marker molecule, proximity of the GPCR with the arrestin may be readily detected. In addition, if the GPCR is conjugated with a marker molecule, compartmentalization of the GPCR with the arrestin may be readily confirmed. The marker molecule used to conjugate with the GPCRs may include those as described above, including, for example, optically detectable marker molecules, such that they may be excited chemically, mechanically, electrically, or radioactively to emit fluorescence, phosphorescence, or bioluminescence. Optically detectable marker molecules may be detected by, for example, immunofluorescence, luminescence, fluorescence, and phosphorescence.

For example, the GPCRs may be antibody labeled with an antibody conjugated to an immunofluorescence molecule or the GPCRs may be conjugated with a luminescent donor. In particular, the GPCRs may be conjugated with, for example, luciferase, for example, Renilla luciferase, or a rhodamine-conjugated antibody, for example, rhodamine-conjugated anti-HA mouse monoclonal antibody. The carboxyl-terminal tail of the GPCR may be conjugated with a luminescent donor, for example, luciferase. The GPCR also may be conjugated with GFP (e.g., at the carboxyl-terminal tail of the GPCR) as described in L. S. Barak et al. “Internal Trafficking and Surface Mobility of a Functionally Intact β₂-Adrenergic Receptor-Green Fluorescent Protein Conjugate”, Mol. Pharm. (1997) 51, 177-184.

Methods of Detection

Methods of detecting the intracellular location, concentration, or translocation of a conjugate of a protein associated with the GPCR desensitization pathway (e.g., an arrestin protein or a GPCR) and a marker molecule or interaction of the conjugate with another molecule (e.g., interaction of an arrestin protein with a GPCR) will vary depending upon the marker molecule(s) used. For example, the methods of detecting the intracellular location, concentration, or translocation of the conjugate of an arrestin protein and a marker molecule or of a conjugate of a GPCR and a marker molecule, including for example, the concentration of arrestin at a cell membrane, colocalization of arrestin with GPCR in endocytic vesicles or endosomes, and concentration of arrestin in clathrin-coated pits, and the like, will vary depending on the marker molecule(s) used. One skilled in the art readily will be able to devise detection methods suitable for the marker molecule(s) used. For optically detectable marker molecules, any optical method may be used where a change in the fluorescence, bioluminescence, or phosphorescence may be measured due to a redistribution or reorientation of emitted light. Such methods include, for example, polarization microscopy, bioluminescence resonance energy transfer (BRET), fluorescence resonance energy transfer (FRET), evanescent wave excitation microscopy, and standard or confocal microscopy.

In one embodiment, an arrestin protein may be conjugated to a GFP and the arrestin-GFP conjugate may be detected by confocal microscopy. In another embodiment, an arrestin protein may be conjugated to a GFP and a GPCR may be conjugated to an immunofluorescent molecule; the conjugates may be detected by confocal microscopy. In an additional embodiment, an arrestin protein may be conjugated to a GFP, and the carboxy-terminus of a GPCR may be conjugated to a luciferase. These conjugates can be detected by BRET. In a further embodiment, an arrestin protein may be conjugated to a luciferase, and a GPCR may be conjugated to a GFP. The luciferase/GFP conjugates may be detected by BRET.

Methods of detection that may be used with the methods of the present invention are also described in U.S. patent application Ser. No. 10/095,620, U.S. Pat. No. 5,891,646 and U.S. Pat. No. 6,110,693, the contents of which are hereby incorporated by reference herein in their entirety.

### EXAMPLES

The invention will be further explained by the following illustrative examples that are intended to be non-limiting.

### Example 1

Different cell lines, each expressing a different GPCR, were pooled and then used to conduct multiplex receptor assays to screen different compounds for GPCR agonist activity. The cells used for the experiment stably expressed α_{1b} adrenergic receptor (α_{1b}AR), beta2-AR (β₂AR), delta opioid receptor (DOR), angiotensin1A receptor (AT_{1A}R), or V2 vasopressin receptor (V2R). Assays pooling three, four, and five of the cell lines were performed. The predominant G protein alpha subunit coupling of the receptors used for the example is as follows: α_{1b}AR-G_{q}; β₂AR-Gₛ; DOR-Gᵢ; AT₁ₐR-G_{q}; V2R-Gₛ.

Cells

The assays were carried out using various “double stable” human osteosarcoma cell (U2OS) lines. Each cell line stably expressed an arrestin-GFP conjugate of the Renilla reniformis green fluorescent protein fused in frame to the carboxyl terminus of rat β-arrestin2 as well as one of the following GPCRs: α_{1b} adrenergic receptor (α_{1b}AR), beta2-AR (α₂AR), delta opioid (DOR) receptor, angiotensin1A receptor (AT_{1A}R), or V2 vasopressin receptor (V2R).

The double stable cell lines were generated using plasmid DNA constructs as described in Oakley et al., “The Cellular Distribution of Fluorescently Labeled Arrestins Provides a Robust, Sensitive, and Universal Assay for Screening G Protein-Coupled Receptors”, Assay and Drug Development Technologies, Volume 1, Number 1-1, pp. 21-30, 2002.

Methods

Osteosarcoma cells (U2OS) stably expressing α_{1b} adrenergic receptor (α_{1b}AR), beta2-AR (β₂AR), delta opioid (DOR), angiotensin1A (AT_{1A}R), or V2 vasopressin (V2R) receptors were maintained and handled following standard cell culture protocol.

Cells were plated 16-24 hours prior to the experiment at approximately 6,000 cells per well. Cells were removed from the flasks with trypsin and suspended in growth media for plating at a density of 100,000 cells per ml.

One of two methods were used to multiplex the cell lines. In one method, each cell line was dispensed independently such that the final cell number was approximately 6,000 cells per well. The dispense volume was adjusted depending on the number of cell lines used. For example, to multiplex three cell lines expressing different GPCRs, 20 μl of each cell line was dispensed per well. In the other method, equal volumes of individual cell lines at 100,000 cells per ml were pooled in 50 ml conical tubes and mixed thoroughly. These pools of cells were then dispensed such that the final cell number was approximately 6,000 cells per well.

To begin the experiment, growth media containing antibiotic and fetal bovine serum (FBS) was removed and replaced with Eagle's minimum essential medium (EMEM)+10 mM HEPES (N-2-Hydroxyethylpiperazine-N′-2-ethanesulfonic acid). Test compounds were added at various concentrations to the wells and the plates were incubated at 37° C. for 45 minutes. Plates were then fixed with 2% formaldehyde containing 1 uM Draq5 nuclear stain.

Plates were imaged for response determination on an In Cell Analyzer 3000, Gen. 1 (Amersham Biosciences), which is a line scanning, confocal imaging system. Pit or vesicle responses were quantitated using the Granularity Module of the Raven Software. That is, the In Cell Analyzer 3000 was used to quantitate the localization of the arrestin-GFP conjugate for the cells in each well. This Granularity Module finds the nucleus of cells and then dilates out a specified distance in which fluorescent spots or “grains” of arrestin-GFP localization are identified based on size and fluorescent intensity. The average of the fluorescent intensity of the identified grains per cell in an acquired image (i.e., Fgrains) was determined for each well on the plates.

Test Compounds

The following test compounds were used in the experiments: norepinephrine (NE) (an alpha-adrenergic selective agonist); angiotensin II (AT), isoproterenol (Iso) (a beta-adrenergic selective agonist); [D-Pen2,D-Pen5]-enkephalin (DPDPE) (a DOR selective agonist); clenbuterol (Clen) (a partial agonist for β₂AR); albuterol (Alb) (a partial agonist for β₂AR); and arginine vasopressin (AVP).

Data

The Fgrains results for the assays of each of the test compounds were plotted versus the concentrations of the respective compounds. Then, using a curve-fitting program, a concentration-response curve was plotted on the graph for compounds that showed agonist activity.

Based on the results of the concentration-response curves, the following data was also obtained for the assays: the change between the fitted maximum and fitted minimum Fgrains value for each compound (i.e., Max Rsp); the compound concentration that caused the half-maximal response (i.e., EC50); the negative log of EC50 (i.e., pEC50); the minimum Fgrains value for each compound as determined by the curve-fitting program (i.e., Min); and the slope of the calculated concentration-response curve. The curve-fitting program allowed the minimum and maximum values as well as the slope and EC50 values to vary rather than fixing the values to specified or collected values.

Controls

Control concentration-response curves were generated for each of the test compounds on each of the cell lines. In order to generate the concentration-response curves, each cell line was plated individually and then the cells were exposed to varying concentrations of each of the test compounds. FIGS. 6A-6E illustrate the control concentration-response curves for each of the compounds for the cell lines expressing α_{1b}AR (FIG. 6A), AT_{1A}R (FIG. 6B), β₂AR (FIG. 6C), DOR (FIG. 6D), and V2R (FIG. 6E). Table I below shows the maximum response, the EC50, the pEC50, and the slope of the concentration-response curves for the test compounds that were agonists for the different receptors.

TABLE I

| Receptor | Test compound | Max Rsp | EC₅₀ (nM) | pEC50 | slope |
|---|---|---|---|---|---|
| α_{1b}AR | NE | 273 | 4.7 | 8.3 | 1.1 |
| AT_{1A}R | AT | 427 | 0.7 | 9.1 | 1.4 |
| β₂AR | Iso | 424 | 0.6 | 9.2 | 1.4 |
| β₂AR | NE | 566 | 1188.6 | 5.9 | 0.9 |
| DOR | DPDPE | 267 | 10.9 | 8.0 | 1.4 |
| V2R | AVP | 443 | 1.6 | 8.8 | 1.7 |

Results

A. Multiplex Receptor Assay With Three GPCRs

The three cell lines expressing β₂AR, AT_{1A}R, and DOR, respectively, were pooled for a multiplex receptor assay according to the protocol described above. The pooled cell lines were exposed to varying concentrations of Iso, AT, and DPDPE. FIG. 7 shows the concentration-response curves resulting from the multiplex receptor assay. Table II below shows the maximum response, the EC50, and the slope of the concentration-response curve for each test compound.

TABLE II

| Test compound | Max Rsp | EC₅₀ (nM) | slope |
|---|---|---|---|
| Iso | 198 | 0.6 | 1.0 |
| AT | 159 | 0.5 | 0.8 |
| DPDPE | 81 | 11.2 | 1.7 |

The EC₅₀ values of the multiplex assay for each test compound were similar to the respective control assays of the individual cell lines for the same compounds. However, the maximum Fgrain response was diminished in the multiplex assay as compared to the control assays.

B. Multiplex Receptor Assay With Three GPCRs Using Partial Agonists

The three cell lines expressing human β₂AR, AT_{1A}R, and DOR, respectively, were pooled for a multiplex receptor assay according to the protocol described above. The pooled cell lines were exposed to varying concentrations of Iso, clenbuterol (Clen), and albuterol (Alb). FIG. 8 shows the concentration-response curves resulting from the multiplex receptor assay. Table III below shows the maximum response, the EC50, and the slope of the concentration-response curve for each test compound.

TABLE III

| Test compound | Max Rsp | EC₅₀ (nM) | slope |
|---|---|---|---|
| Iso | 209 | 1.9 | 0.6 |
| Clen | 148 | 45.9 | 0.5 |
| Alb | 93 | 1.5 | 0.5 |

The EC₅₀ values of the multiplex assay for each test compound were similar to the respective control assays of the individual cell lines for the same compounds. However, the maximum Fgrain response was diminished in the multiplex assay as compared to the control assays.

C. Multiplex Receptor Assay With Four GPCRs

The four cell lines expressing α_{1b}AR, β₂A, AT_{1A}R, and DOR, respectively, were pooled for a multiplex receptor assay according to the protocol described above. The pooled cell lines were exposed to varying concentrations of Iso, AT, NE, and DPDPE. FIG. 9 shows the concentration-response curves resulting from the multiplex receptor assay. As shown in FIG. 9, the response at the two highest concentrations of NE were not used for the analysis due to the loss of selectivity with respect to β₂AR (as determined in the control plates). Table IV below shows the maximum response, the EC50, and the slope of the concentration-response curve for each test compound.

TABLE IV

| Test compound | Max Rsp | EC₅₀ (nM) | slope |
|---|---|---|---|
| Iso | 146 | 0.5 | 1.7 |
| AT | 114 | 0.5 | 1.2 |
| NE | 62 | 9.7 | 1.2 |
| DPDPE | 74 | 39.4 | 0.8 |

The EC₅₀ values of the multiplex assay for each test compound were again similar to the respective control assays of the individual cell lines for the same compounds and the maximum Fgrain response was again diminished in the multiplex assay as compared to the control assays.

D. Multiplex Receptor Assay With Five GPCRs

The five cell lines expressing α_{1b}AR, β₂AR, AT_{1A}R, DOR, and V2R, respectively, were pooled for a multiplex receptor assay according to the protocol described above. The pooled cell lines were exposed to varying concentrations of Iso, AT, NE, DPDPE, and AVP. FIG. 10 shows the concentration-response curves resulting from the multiplex receptor assay. Table V below shows the maximum response, the EC50, and the slope of the concentration-response curve for each test compound.

TABLE V

| Test compound | Max Rsp | EC₅₀ (nM) | pEC50 | slope |
|---|---|---|---|---|
| Iso | 128 | 0.7 | 9.2 | 1.2 |
| AVP | 58 | 0.5 | 9.3 | 2.5 |
| AT | 93 | 0.7 | 9.2 | 1.2 |
| NE | 15 | 300 | 6.5 | 2.2 |
| DPDPE | 52 | 20 | 7.7 | 1.0 |

The EC₅₀ values of the multiplex assay for each test compound were again similar to the respective control assays of the individual cell lines for the same compounds and the maximum Fgrain response was again diminished in the multiplex assay as compared to the control assays.

### Example 2

### Multiplex Rector Assay With Three GPCRs Using LOPAC 640 Library

The three cell lines expressing human α_{1b}AR, β₂AR, and DOR, respectively, were pooled for a multiplex receptor assay according to the protocol described in Example 1 above. Wells containing the pooled cell lines were separately exposed to one of the compounds contained in the LOPAC 640 Library of Pharmaceutically-Active Compounds (Sigma-Aldrich) so that all of the compounds were tested. The three cell lines were also plated individually and wells were separately exposed to one of the compounds contained in the LOPAC 640 library so that all of the compounds were tested against each of the separate individual cell lines.

FIG. 11 is a table showing the individual and multiplex assay responses to the compounds in the LOPAC 640 library. Only those compounds exhibiting agonist activity in the individual and/or multiplexed assays are listed in FIG. 11. The individual assay responses (i.e., α_{1b}AR, β₂AR, and DOR) and multiplex assay responses (i.e., multi) are expressed as percentage maximal response to control agonist stimulation. Eleven compounds (i.e., 1.7% of the compounds in the library) had activity in the multiplex assay but no activity in the cell lines plated individually, while there were 16 compounds (i.e., 2.5% of the compounds in the library) showing activity in the cell lines plated individually that did not show a response in the multiplex assay. Wells with a response of greater than three times the standard deviation (used as an indication of GPCR agonist activity for this experiment with the LOPAC 640 library) are listed in FIG. 11 using bold text.

FIG. 12 lists the results of the subset of adrenergic agonists from the LOPAC 640 library. As in FIG. 11, the responses are expressed as percentage maximal response to control agonist stimulation, and wells with a response of greater than three times the standard deviation are listed in bold text.

### Example 3

### Multiplex Assay Using “spotting” of Compounds on 384 Well Plate

The three cell lines expressing α_{1b}AR, β₂AR, and AT_{1A}R, respectively, were pooled for a multiplex receptor assay according to the protocol described in Example 1 above. The cells were plated on a 384 well plate at 2000 cells of each cell line per well. Varying concentrations of isoproterenol, angiotensin, and norepinephrine were randomly distributed in a blinded fashion on the 384 well plate, and a response was calculated for each well on the plate.

FIG. 13 is a representation of a portion of the 384 well plate. In FIG. 13, each number represents a response value assigned to an individual well as a result of the assay, and wells to which one of the test agonists were added are enclosed in a box. As shown in FIG. 13, varying concentrations of isoproterenol were “spotted” randomly in the wells of rows F, G, and H; varying concentrations of angiotensin were “spotted” randomly in the wells of rows J, K, and L; and varying concentrations of norepinephrine were “spotted” randomly in the wells of rows N, O, and P. Wells with a response to the individual compounds greater than three times the standard deviation (used as an indication of GPCR agonist activity for this experiment) are listed in FIG. 13 as follows: isoproterenol=*, angiotensin=**, and norepinephrine=***. Wells in which agonist was added but no indication of GPCR agonist activity was detected (i.e., those wells having a box but no asterisks) represent those wells to which low concentrations of agonist were added. The results illustrate that wells with indications of GPCR agonist activity are able to be determined after spotting of the compounds randomly on the plate.

While the invention has been described in detail and with reference to specific embodiments thereof, it will be apparent to one skilled in the art that various changes and modifications can be made without departing from the spirit and scope of the invention.

1-47. (canceled)

48. A method of screening a test composition for G protein-coupled receptor (GPCR) agonist activity against multiple GPCRs, comprising:
(a) providing a mixture of cells comprising a first cell and a second cell, said first cell comprising a first GPCR and a first conjugate, said second cell comprising a second GPCR and a second conjugate;
(b) exposing said mixture of cells to a test composition;
(c) detecting said first and second conjugates for an indication of GPCR agonist activity.

49. The method of claim 48, wherein at least one conjugate comprises a marker molecule and a protein.

50. The method of claim 49, wherein the marker molecule comprises a radioisotope, enzyme, fluorescent group, chemiluminescent group or any combination thereof.

51. The method of claim 49, wherein the marker molecule comprises a epitope tag, affinity label or combination thereof.

52. The method of claim 49, wherein said marker molecule is a fluorescent protein.

53. The method of claim 52, wherein said fluorescent protein is green fluorescent protein (GFP).

54. The method of claim 49, wherein said protein is an arrestin.

55. The method of claim 48, wherein said first and second conjugates are the same.

56. The method of claim 48, wherein said first and second GPCRs interact with different G_{α} protein subunits.

57. The method of claim 48, wherein said indication of GPCR agonist activity is translocation or localization of at least one conjugate.

58. The method of claim 48, wherein said indication of GPCR agonist activity is translocation or localization of at least one conjugate to a vesicle, endosome, granule or pit.

59. The method of claim 48, wherein said indication of GPCR agonist activity is an increase in translocation or localization of at least one conjugate, after exposure said mixture of cells to said test composition.

60. The method of claim 48, wherein said indication of GPCR agonist activity is an increased level of translocation or localization of at least one conjugate, with respect to a predetermined level of translocation or localization of at least one conjugate.

61. A method of screening a test composition for G protein-coupled receptor (GPCR) agonist activity against multiple GPCRs, comprising:
(a) providing a cell comprising a first GPCR, a first conjugate associated with the desensitization pathway of said first GPCR, a second GPCR different from said first GPCR, and a second conjugate associated with the desensitization pathway of said second GPCR;
(b) exposing said cell to a test composition;
(c) detecting said first and second conjugates for an indication of GPCR agonist activity.

62. The method of claim 61, wherein at least one conjugate comprises a marker molecule and a protein.

63. The method of claim 62, wherein the marker molecule comprises a radioisotope, enzyme, fluorescent group, chemiluminescent group or any combination thereof.

64. The method of claim 62, wherein the marker molecule comprises an epitope tag, affinity label, or combination thereof.

65. The method of claim 62, wherein said marker molecule is a fluorescent protein.

66. The method of claim 65, wherein said fluorescent protein is green fluorescent protein (GFP).

67. The method of claim 62, wherein said protein is an arrestin.

68. The method of claim 61, wherein said first and second conjugates are the same.

69. The method of claim 61, wherein said first and second GPCRs interact with different G_{α} protein subunits.

70. The method of claim 61, wherein said indication of GPCR agonist activity is translocation or localization of at least one conjugate.

71. The method of claim 61, wherein said indication of GPCR agonist activity is translocation or localization of at least one conjugate to a vesicle, endosome, granule or pit.

72. The method of claim 61, wherein said indication of GPCR agonist activity is an increase in translocation or localization of at least one conjugate, after exposure of said cell to said test composition.

73. The method of claim 61, wherein said indication of GPCR agonist activity is an increased level of translocation or localization of at least one conjugate, with respect to a predetermined level of translocation or localization of at least one conjugate.
